(19) United States
(12) Patent Application Publication
(10) Pub. No.: US 2015/0209407 A1
(43) Pub. Date: Jul. 30, 2015

(54) METHODS FOR THE TREATMENT AND PREVENTION OF OSTEOPOROSIS AND BONE-RELATED DISEASES

(71) Applicant: THE TRUSTEES OF THE UNIVERSITY OF PENNSYLVANIA, Philadelphia, PA (US)

(72) Inventor: ROBERT J. PIGNOLO, Kennett Square, PA (US)

(73) Assignee: THE TRUSTEES OF THE UNIVERSITY OF PENNSYLVANIA, Philadelphia, PA (US)

(21) Appl. No.: 14/682,892

(22) Filed: Apr. 9, 2015

Related U.S. Application Data
(63) Continuation of application No. PCT/US13/64208, filed on Oct. 10, 2013.
(60) Provisional application No. 61/712,427, filed on Oct. 11, 2012.

(51) Int. Cl. A61K 38/17; G01N 33/68; C07K 14/47

(52) U.S. Cl. CPC A61K 38/1709; C07K 14/4705; G01N 33/6893; C07K 2319/30; G01N 2333/4706; G01N 2800/108; A61K 48/00

(57) ABSTRACT

The present disclosure is directed to the identification that R-Spondin-1 (RSpo1) acts as a positive regulator of bone formation. Accordingly, the presently disclosed subject matter provides methods for treating and/or preventing osteoporosis or bone-related disorders, including administering a pharmaceutical composition of a therapeutically effective amount of an R-Spondin (RSpo) agonist, e.g., an RSpo protein or functional fragment thereof, peptidomimetic, nucleic acid, small molecule, or other drug candidate, to a subject, e.g., a mammal. In one exemplary embodiment, the RSpo agonist is a therapeutic vector including a nucleic acid molecule encoding RSpo protein or a functional fragment thereof. The presently disclosed subject matter also provides methods for diagnosing osteoporosis and other bone-related disorders using RSpo as a marker, as well as screening methods for identifying other proteins that are involved in bone formation.

### CROSS-REFERENCE TO RELATED APPLICATIONS

This application is a continuation of PCT/US13/064208, filed Oct. 10, 2013, and which claims priority to U.S. Provisional Application No. 61/712,427, filed Oct. 11, 2012, both of which ares hereby incorporated by reference in their entireties.

### GRANT INFORMATION

This invention was made with government support under Grant No. R01AG028873 awarded by National Institutes of Health. The government has certain rights in the invention.

### SEQUENCE LISTING

The instant application contains a Sequence Listing which has been submitted in ASCII format via EFS-Web and is hereby incorporated by reference in its entirety. The ASCII copy, created on Oct. 8, 2013, is named 08140601.txt and is 19,300 bytes in size.

### BACKGROUND

According to the National Osteoporosis Foundation, osteoporosis is a major public health threat for an estimated 44 million Americans, or 55 percent of people 50 years of age and older. In the U.S., 10 million individuals are estimated to already have the disease and almost 34 million more are estimated to have low bone density, placing them at increased risk for osteoporosis and broken bones.

Bone tissue adapts to its functional environment by optimizing its morphology for mechanical demand. Mechanosensitive cells that recognize and respond to forces in the skeleton include mesenchymal progenitor cells (MPCs), osteoblasts, osteoclasts, osteocytes and cells of the vasculature.¹ Proximal mechanosensing mechanisms can involve ion channels, integrins, connexins, caveolar and noncaveolar lipid rafts, as well as cell shape alteration at the membrane or cytoskeleton.¹ G-proteins, MAPKs, and nitric oxide have been implicated in downstream intracellular signaling.¹

The skeleton's sensitivity to mechanical stimuli represents a critical determinant of bone mass and physical activity is an important strategy to reduce osteoporosis and fractures in the elderly. Increases in bone mineral density (BMD) or reductions in bone loss can occur with sufficient exercise or mechanical loading.^{2,3} Despite potentially missed opportunities to maintain a strong skeleton into adulthood and old age, and minimizing bone loss in peri-menopausal years and later life, physical activity and exercise at virtually any age can still increase BMD and potentially reduce fracture risk with minimal therapeutic harm.³⁻⁵ Low level mechanical stimuli can improve both the quantity and the quality of trabecular bone,⁶ are anabolic to trabecular bone in children,⁷ and increase bone and muscle mass in the weight-bearing skeleton of young adult females with low BMD.⁸ Therefore, the ability to use mechanical signals to improve bone health through exercise and devices that deliver mechanical signals can be an approach to age-related bone loss; however, the extracellular or circulating mediators of such signals are largely unknown.

### SUMMARY

The presently disclosed subject matter utilizes certain proteins, including R-Spondin 1 (RSpo1; a Wnt pathway modulator), which are induced and secreted by mesenchymal progenitor cells (MPCs) in response to low magnitude mechanical signals (LMMS). It has been shown that these mechanical signals can increase MPCs as well as their ability to differentiate into osteoblasts, a mediator of new bone formation. As described herein, RSpo1 was elevated 28-fold in LMMS stimulated MPCs as compared to RSpo1 secreted by unstimulated, control MPCs, indicating that RSpo1 is involved in the response to mechanical signals that can promote bone formation. In addition, expression of R-Spondin family members, R-Spondin-2 and R-Spondin-4, was elevated in vibration-stimulated MPCs as compared to unstimulated, control MPCs, indicating that R-Spondin-2 and R-Spondin-4 are also involved in the response to mechanical signals that can promote bone formation.

Furthermore, as described herein, RSpo1 acts to promote bone formation and is capable of increasing bone mineral apposition in mammals in vivo. Accordingly, the present disclosure provides methods for anabolically increasing bone formation, preventing bone loss, and improving bone mass using one or more proteins induced in response to stimuli, e.g., mechanical signals such as LMMS, i.e., “vibration-induced bone-enhancing” or “vibe” proteins, including RSpo1 and R-Spondin family members having similar structure and activity, e.g., R-Spondin-2 and R-Spondin-4. Additional non-limiting examples of vibe proteins include Tissue inhibitor of metalloproteinases (TIMPs), Plasminogen activator inhibitor-1 (PAI-1) precursor, Collagen alpha 1 chain precursor variant, Fibrillin-1 precursor, and unidentified protein products, NCBI GI:62822120, GI:189053417, GI:189055325, GI:158258302, and GI:158256710.

In certain embodiments, the disclosure provides methods for treating, preventing or delaying the onset of osteoporosis or bone-related disorders, comprising administering a pharmaceutical composition comprising a therapeutically effective amount of a vibe agonist, such as, but not limited to, an RSpo1 agonist or an R-Spondin (RSpo) family member agonist having similar activity and structure, e.g., an RSpo (e.g., RSpo1) protein or functional fragment thereof, peptidomimetic, nucleic acid, small molecule, or other drug candidate, to a subject, e.g., a human patient. In certain embodiments, the RSpo agonist is an RSpo protein (e.g., RSpo1), or functional fragment thereof, fused to the Fc portion of an antibody, or a portion thereof to increase half-life or bioavailability, or fused to a skeletal delivery molecule (e.g., a bisphosphonate) to target tissue specificity. In certain embodiments, the Rspo (e.g., RSpo1) agonist is a therapeutic vector including a nucleic acid molecule encoding an RSpo protein or a functional fragment thereof.

In certain embodiments, the vibe agonist is administered alone or in combination with another agent used for the treatment or prevention of osteoporosis or bone-related disorders or symptoms thereof. In certain embodiments, the method of the presently disclosed subject matter further includes administering a second agent for the treatment or prevention of osteoporosis or a related bone disorder, e.g., a drug product or nutritional supplement. For example, the agonist of the disclosure can be administered in combination with calcium, vitamin D, bisphosphonates, such as, for example, Alendronate (FOSAMAX®), Risedronate (ACTONEL®, ATELVIA®), Ibandronate (BONIVA®), and Zoledronic acid (RECLAST®, ZOMETA®), hormone related therapy such as Raloxifene (EVISTA®), or other drugs such as Teriparatide (FORTEO®) or Denosumab (PROLIA®, XGEVA®).

The disclosure further provides methods for identifying subjects that have an elevated risk for developing osteoporosis or a bone-related disorder, or are suffering from osteoporosis or a bone-related disorder. Diagnostic methods include, for example, measuring levels and/or activity of an RSpo gene or protein, or another vibe protein or gene, where decreased level and/or activity indicates that the subject is at risk for or suffering from bone loss or osteoporosis or a bone-related disorder. Optionally, the subject is subsequently treated for the osteoporosis or a bone-related disorder.

In another aspect, the disclosure provides screening methods for identifying additional proteins that are up- or down-regulated in response to stimuli, e.g., LMMS. Proteins identified using the screening methods described herein are candidate targets for modulation of bone formation and use in preventing and treating osteoporosis and bone-related disorders.

### BRIEF DESCRIPTION OF THE FIGURES

FIG. 1A-B. Vibration (LMMS)-induced expression of secreted RSpo1. (a) Rspo1 Western blot analysis of secreted protein (supernatant) and whole cell protein (cell lysate) produced in response to LMMS. (b) RT-PCR analysis of the RSpo1 transcript.

FIG. 2. Senescent MPCs do not secrete RSpo1 in response to LMMS. Shown are Western blot analyses of secreted protein (supernatant) and whole cell protein (cell lysate) produced by senescent (Sen) and early passage (EP) MPCs using antibodies against Rspo1, type I collagen (Col 1), and β-actin.

FIG. 3. Rspo1 enhances bone formation in three mouse models of age-related bone loss. Animals used were 18.5 month old wild-type mice (WT; n=4 per group), 6 month old Terc^{-/-} single mutants (n=3 per group) and 3-month old Wrn^{-/-} Terc^{31 /-} double mutants (n=3 per group). *p<0.05; **p<0.02.

FIG. 4. Provides an algorithm outlining an exemplary screening method for the identification of secreted vibratory-induced bone-enhancing (vibe) proteins.

FIG. 5A-B. (a) Exemplary human R-Spondin 1 (SEQ ID NO:1), R-Spondin-2 (SEQ ID NO:3), and R-Spondin-4 (SEQ ID NO:5) nucleic acid sequences. (b) Exemplary human R-Spondin 1 (SEQ ID NO:2), R-Spondin-2 (SEQ ID NO:4), and R-Spondin-4 (SEQ ID NO:6) amino acid sequences.

FIG. 6A-F. Phenotype of human mesenchymal progenitor cells (MPCs). MPCs are CD 73+ CD90+ CD105+ CD45—cells as determined by flow cytometric analysis (a-d). MPCs are capable of differentiating into osteoblasts and adipocytes in vitro (e, f).

FIG. 7A-B. Vibration (LMMS)-induced expression of Rspo1. (a) Rspo1 is recognized as a protein of ˜30 kD by Western blot analysis of whole cell protein. (b) The circulating level of Rspo1 is increased in response to LMMS in a 29 year-old healthy male.

FIG. 8. Rspo1 promotes bone turnover and bone formation in mouse models of osteoporosis. Animals used were 18.5 month old wild-type mice (WT; n=4 per group), 6 month old Terc−/− single mutants (n=3 per group) and 3-month old Wrn−/− Terc−/− double mutants (n=3 per group). N.Ob/BS, number of osteoblasts/bone surface; N.OC/BS, number of osteoclasts/bone surface; BV/TV, bone volume/total volume; OS/BS, osteoid surface/bone surface. *, p<0.05; **, p<0.02, ***, p<0.005.

FIG. 9A-B. Vibration (LMMS)-induced expression of secreted RSpo2 and RSpo4. (a) RT-PCR analysis of the RSpo2 transcript (n=3 per group). (b) RT-PCR analysis of the RSpo4 transcript (n=3 per group). *p<0.05.

### DETAILED DESCRIPTION

Mesenchymal progenitor cells (MPCs) can differentiate into cells that form mesodermal tissues such as bone and fat. Low magnitude mechanical signals (LMMS) have been shown to increase the number of MPCs, as well as their potential to differentiate into osteoblasts versus adipocytes.⁹ The number and activity of osteoblasts responsible for synthesizing new bone matrix are substantially reduced with aging, and limitations in the ability of osteogenic precursors to replace osteoblasts can potentially explain many aspects of age-related bone loss.¹⁰⁻¹⁸ This disclosure describes the identification of secreted proteins up-regulated by vibratory stimulation of MPCs (“vibration-induced bone-enhancing” or “vibe” proteins), including, but not limited to, R-Spondin-1 (RSpo-1) and R-Spondin family members, R-Spondin-2 and R-Spondin-4. Additional non-limiting examples of vibe proteins include Tissue inhibitor of metalloproteinases (TIMPs), Plasminogen activator inhibitor-1 (PAI-1) precursor, Collagen alpha 1 chain precursor variant, Fibrillin-1 precursor, and unidentified protein products, NCBI GI:62822120, 61:189053417, GI:189055325, GI:158258302, and GI:158256710.

As described herein, the vibe protein RSpo-1 has been shown to have the capacity to promote bone formation in three mouse models of age-related bone loss. Thus, vibe genes serve as extracellular mediators of mechanical signals and modulation of such genes and proteins can result in modulation (e.g., increase) in bone formation in mammalian subjects.

In one aspect, the present disclosure provides methods of treating or preventing osteoporosis or a bone-related disorder, where the method includes treating a patient in need with a therapeutically effective amount of a vibe agonist, such as but not limited to, an RSpo1 agonist or an RSpo family member agonist having similar structure and activity to RSpo1, e.g, R-Spondin-2 and R-Spondin-4. The present disclosure also provides methods for anabolically increasing bone formation, slowing down the decrease of bone mineral density, increasing bone mineral density, or increasing bone mass in a patient in need of such treatment, the method of which includes treating the patient with a vibe agonist, such as but not limited to, an RSpo1 agonist or an RSpo family member agonist having similar activity and structure, in an amount sufficient to anabolically increase bone formation, slow down the decrease of bone mineral density, increase bone mineral density, or increase bone mass content. In certain embodiments, the present disclosure provides methods of treating or preventing osteoporosis or a bone-related disorder, where the method includes treating a patient in need with a therapeutically effective amount of one or more vibe proteins or genes.

As used herein, a “vibe agonist” includes agents that increase the expression or activity of a vibe gene or protein. As used herein, an “RSpo agonist” includes an agent that increases the expression or activity of an RSpo1 gene or protein or other RSpo family member having similar structure and activity, e.g, R-Spondin-2 (RSpo2) and R-Spondin-4 (RSpo4). An “RSpo agonist” includes the RSpo protein itself (e.g., RSpo1, RSpo2, RSpo4). For example, an RSpo agonist includes an RSpo protein or functional fragment thereof, peptidomimetic, nucleic acid, small molecule, or other drug candidate. In one embodiment, the RSpo agonist is an RSpo protein, or functional fragment thereof. For example, the RSpo agonist is RSpo1 protein, or a functional fragment thereof. In certain embodiments, the RSpo protein can be fused to a skeletal delivery molecule (e.g., a bisphosphonate), or a portion thereof. In certain embodiments, the RSpo agonist is a therapeutic vector including a nucleic acid molecule encoding an RSpo protein or a functional fragment thereof. For example, the RSpo agonist can be a therapeutic vector including a nucleic acid molecule encoding RSpo1 protein or a functional fragment thereof.

In certain embodiments, the RSpo agonist is an R-Spondin family member, which has similar activity to RSpo1 has, e.g., the ability to slow down the decrease of bone mineral density, increase bone mineral density, or increase bone mass, and acts anabolically to increase bone formation. For example, but not by way of limitation, R-Spondin family members having similar activity or structure include R-Spondin-1, R-Spondin-2 and R-Spondin 4, but excludes R-Spondin 3. In certain embodiments, the RSpo agonist is R-Spondin-2. In certain embodiments, the RSpo agonist is R-Spondin-4.

In certain embodiments, the vibe agonist, such as but not limited to, an RSpo agonist, is administered alone. In certain embodiments, one or more vibe agonists are administered. For example, but not by way of limitation, R-Spondin-1, R-Spondin-2, R-Spondin-4, or combinations thereof, can be administered. In certain embodiments, the vibe agonist is administered in combination (either concurrently or sequentially) with a second agent for the treatment or prevention of osteoporosis or a related bone disorder, e.g., a drug product or nutritional supplement. For example, the agonist of the present disclosure can be administered in combination with calcium, vitamin D, bisphosphonates, such as, for example, Alendronate (FOSAMAX®), Risedronate (ACTONEL®, ATELVIA®), Ibandronate (BONIVA®), and Zoledronic acid (RECLAST®, ZOMETA®), hormone related therapy such as Raloxifene (EVTSTA®), or other drugs such as Teriparatide (FORTE®) or Denosumab (PROLIA®, XGEVA®).

The term “osteoporosis” is defined by the World Health Organization as “ . . . a systemic skeletal disease characterized by low bone mass and micro-architectural deterioration of bone tissue, with a consequent increase in bone fragility and susceptibility to fracture” (WHO Consensus Development Conference 1993). The clinical definition of osteoporosis is a condition in which the bone mineral density (BMD) or bone mineral concentration (BMC) is greater than about 2.5 standard deviations (SD) below the mean of young healthy women. Severe osteoporosis is defined as having a BMD or BMC greater than about 2.5 SD below the mean of young healthy women and the presence of one or more fragility fractures. Since bone loss is not strictly confined to specific sites, osteoporosis can manifest itself in various ways including alveolar, femoral, radial, vertebral or wrist bone loss or fracture incidence, postmenopausal bone loss, severely reduced bone mass, fracture incidence or rate of bone loss.

As used herein, “osteoporosis or bone-related disorders” includes osteoporosis as well as, for example, osteopenia. Osteopenia is commonly defined as a BMD between −1.0 and −2.5, but other criteria can be used to identify the condition. In addition, “bone-related disorders” also include localized bone loss, e.g., associated with periodontal disease, with bone fractures, or with periprosthetic osteolysis. Non-limiting examples of additional bone-related disorders include Paget's disease, hyperthyroidism, hyperparathyroidism, osteomalacia, chronic renal failure, Cushing's syndrome, and various cancers, including both osteogenic cancers (e.g., osteochondromas and osteogenic sarcomas) and non-osteogenic cancers that have metastasized to bone tissue. These and other bone-related disorders are known in the art, and have been reviewed, for example in Boyce et al. (1999, Lab. Invest. 79:83-94) and Berkow et al. (Eds., 1992, The Merck Manual, Sixth Edition, Merck & Co., Inc., Rahway, N.J.) (the contents of which are incorporated herein by reference).

In certain embodiments of the various methods of the presently disclosed subject matter, a step of identifying a patient in need of treatment or prevention can be optionally included. In certain embodiments, the disclosure also provides methods for identifying subjects that have an elevated risk for developing osteoporosis or a bone-related disorder, or are suffering from osteoporosis or a bone-related disorder, by measuring levels and/or activity of a vibe gene or protein, where decreased level and/or activity indicates that the subject is at risk for or suffering from bone loss or osteoporosis or a bone-related disorder. In certain embodiments, the methods for identifying subjects that have an elevated risk for developing osteoporosis or a bone-related disorder, or are suffering from osteoporosis or a bone-related disorder, includes measuring levels and/or activity of an RSpo gene or protein, where decreased level and/or activity indicates that the subject is at risk for or suffering from bone loss or osteoporosis or a bone-related disorder. In certain embodiments, the subject can be subsequently treated for the osteoporosis or a bone-related disorder. For example, but not by way of limitation, a method of the presently disclosed subject matter can include the identification of a patient that is at risk for or suffering from osteoporosis or a bone-related disorder followed by treatment of the patient with a vibe agonist.

Other methods for identifying patients that have osteoporosis or bone-related disorders are known in the art. For example, the bone mineral density (BMD) of a patient can be determined using, e.g., dual energy X-ray absorptiometry (DXA or DEXA), serum markers, X-rays, etc.

Also, the identification of patients at risk of developing osteoporosis or generalized or local bone loss is generally known in the art. For example, patients having risk factors that are typically associated with an increased likelihood of bone loss and of developing osteoporosis can be identified. Osteoporosis or bone-related disorders can be associated with or caused by any risk factor, such as, for example, age, gender, family history, body type, ethnicity, dietary insufficiencies, such as calcium or vitamin D insufficiency, hormonal imbalance, anorexia nervosa, certain medications (e.g., steroids, glucocorticoid and anticonvulsants), immobilization, smoking, alcohol use, or other secondary causes including various disease states (e.g., rheumatoid arthritis, osteomalacia, Paget's disease, periodontal disease, bone fracture, and periprosthetic osteolysis and gastrointestinal diseases). In addition, patients having certain types of cancer (e.g., lung cancer, breast cancer, prostate cancer, multiple myeloma or neuroendocrine tumors) with or without bone metastasis, and patients undergoing hormone ablation therapy for either prostate or breast cancer, are all at risk of bone loss, bone fractures, increased frequency of skeletal-related events, and osteoporosis.

### Vibe Agonists

The term “Vibe therapeutic” refers to various forms of vibe polypeptides, as well as peptidomimetics, nucleic acids, or small molecules, which can slow down the decrease of bone mineral density, increase bone mineral density, or increase bone mass or treat or prevent osteoporosis or a bone-related disorder in a subject. A vibe therapeutic that mimics or potentiates the activity of a wild-type vibe polypeptide, such as, but not limited to, RSpo polypeptide (e.g., RSpo1, RSpo2 or RSpo4) is a “vibe agonist.”

The term “agonist,” as used herein, is meant to refer to an agent that mimics or upregulates (e.g., potentiates or supplements) vibe protein (e.g., RSpo1) bioactivity. For example, but not by way of limitation, an RSpo agonist can be a wild-type RSpo (e.g., RSpo1) protein, derivative, or functional fragment thereof, having at least one bioactivity of the wild-type RSpo (e.g., RSpo1) and the ability to slow down the decrease of bone mineral density, increase bone mineral density, or increase bone mass, in a subject. An RSpo (e.g., RSpo1) agonist can also be an agent that upregulates expression of an RSpo (e.g., RSpo1) gene. An agonist can also be a compound which increases the interaction of an RSpo1 polypeptide with another molecule, e.g., a target peptide such as an RSpo (e.g., RSpo1) receptor. Accordingly, an RSpo (e.g., RSpo1) agonist can include a peptidomimetic, protein, or functional fragment thereof, peptide, nucleic acid (e.g., using adenoviral expression), small molecule (or other drug candidate) that increases the expression or activity of RSpo (e.g., RSpo1). In certain embodiments, the RSpo1 agonist is an analog of RSpo (e.g., RSpo1).

“Peptide mimetics” or “peptidomimetics” are described in Fauchere, J. (1986) Adv. Drug Res. 15:29; Veber and Freidinger (1985) TINS p.392; and Evans et al. (1987) J. Med. Chem 30:1229. Peptide mimetics that are structurally similar to therapeutically useful peptides can be used to produce an equivalent therapeutic effect. Generally, peptidomimetics are structurally similar to a paradigm polypeptide (i.e., a polypeptide that has a biological or pharmacological activity), but have one or more peptide linkages replaced by a linkage selected from the group consisting of: —CH₂NH—, —CH₂S—, —CH₂CH₂2—, —CH=CH-(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH₂NH—, CH₂CH₂—); Spatola, A. F. et al., Life Sci (1986) 38:1243-1249 (—CH₂S); Hann, M. M., J. Chem Soc Perkin Trans I (1982) 307-314 (—CH—CH—, cis and trans); Almquist, R. G. et al., J Med Chem (1980) 23:1392-1398 (—COCH₂—); Jennings-White, C. et al., Tetrahedron Lett (1982) 23:2533 (—COCH₂—); Szelke, M. et al., European Appln. EP 45665 (1982) CA: 97:39405 (1982) (—CH(OH)CH₂—); Holladay, M. W. et al., Tetrahedron Lett (1983) 24:4401-4404 (—C(OH)CH₂—); and Hruby, V. J., Life Sci (1982) 31:189-199 (—CH₂S—).

Peptide mimetics can have significant advantages over polypeptide embodiments, including, for example: more economical production; greater chemical stability; enhanced pharmacological properties (half-life, absorption, potency, efficacy, etc.); altered specificity (e.g., a broad-spectrum of biological activities); reduced antigenicity; and others.

Vibe Polypeptide Agonists

The term “vibe protein” is intended to encompass polypeptides having full-length sequences as well as fragments and variants thereof. For example, but not by way of limitation, the terms “RSpo1 polypeptide,” “RSpo1 protein” and “RSpo1” are intended to encompass polypeptides having the exemplary amino acid sequence of SEQ ID NO:2 (Genbank accession number ABA54597), fragments thereof (e.g., functional fragments thereof), and variants thereof, and include agonist polypeptides. In certain embodiments, RSpo1 is a 70 kDa, 263 amino acid secreted protein. An RSpo polypeptide can also include R-Spondin-2, which has an exemplary amino acid sequence set forth as SEQ ID NO:4 (Genbank accession number NP_{—}848660), and R-Spondin 4, which has an exemplary amino acid sequence set forth as SEQ ID NO:6 (Genbank accession number NP_{—}001025042). It is to be understood that the RSpo1, RSpo2, and RSpo4 amino acid sequences set forth above are exemplary amino acid sequences. The disclosure expressly includes all fragments, variants and isoforms of these and other vibe proteins.

In certain embodiments, the presently disclosed subject matter provides for the use of an isolated or purified vibe polypeptide, such as, but not limited to an RSpo (e.g., RSpo1) polypeptide and variants and fragments thereof. The presently disclosed subject matter also encompasses the use of sequence variants. Variants include a substantially homologous protein encoded by the same genetic locus in an organism, i.e., an allelic variant. Variants also encompass proteins derived from other genetic loci in an organism, but having substantial homology to the particular vibe polypeptide, such as but not limited to, the RSpo protein of SEQ ID NO: 2, 4, or 6. Variants also include proteins substantially homologous to the vibe protein but derived from another organism, i.e., an ortholog. Variants also include proteins that are substantially homologous to the particular vibe protein and are produced by chemical synthesis. Variants also include proteins that are substantially homologous to the particular vibe protein and are produced by recombinant methods.

As used herein, two polypeptides (or regions thereof) are substantially homologous when the amino acid sequences are at least about 60-65%, 65-70%, 70-75%, 80-85%, 90-95%, or 95-99% or more homologous. In certain embodiments, two polypeptides (or regions thereof) are substantially homologous when the amino acid sequences are at least about 90-95% or more homologous. In certain embodiments, a substantially homologous amino acid sequence, according to the presently disclosed subject matter will be encoded by a nucleic acid sequence hybridizing to the nucleic acid sequence, or portion thereof, of the sequence shown in SEQ ID NO: 2, 4, or 6 under stringent conditions.

The vibe proteins (e.g., RSpo1) used in the methods of the presently disclosed subject matter can also include vibe polypeptides having additions, deletions or substitutions of amino acid residues (variants) which do not substantially alter the biological activity of the protein. Those individual sites or regions of a vibe protein, such as RSpo which can be altered without affecting biological activity can be determined by examination of the structure of the RSpo binding domains, for example. Alternatively, one may empirically determine those regions which would tolerate amino acid substitutions by alanine scanning mutagenesis (Cunningham et al. Science 244, 1081-1085 (1989)). In this method, selected amino acid residues are individually substituted with a neutral amino acid (e.g., alanine) in order to determine the effects on biological activity.

It is generally recognized that conservative amino acid changes are least likely to perturb the structure and/or function of a polypeptide. Accordingly, the presently disclosed subject matter encompasses one or more conservative amino acid changes within a vibe protein, e.g., an RSpo protein. Conservative amino acid changes generally involve substitution of one amino acid with another that is similar in structure and/or function (e.g., amino acids with side chains similar in size, charge and shape). Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residue within a vibe protein can be replaced with other amino acid residues from the same side chain family and the altered protein can be tested for retained function using the functional assays described herein.

Modifications can be introduced into an antibody used in the methods of this disclosure, e.g., a diagnostic method of this disclosure, by standard techniques known in the art, such as site-directed mutagenesis and PCR-mediated mutagenesis, provided that activity, e.g., binding activity, and affinity, is retained.

The presently disclosed subject matter also provides for fusion proteins including vibe proteins and compositions thereof. In certain embodiments, the vibe agonist of the present disclosure can be fused to a skeletal delivery molecule (e.g., a bisphosphonate) for bone-specific delivery of the RSpo agonist (as described in, for example, Hirabayashi et al. Clinical Pharmacokinetics, 42:15; 1319-1330, the contents of which are expressly incorporated herein by reference).

In certain embodiments, the presently disclosed subject matter provides for fusion proteins of vibe protein, or functional fragments thereof, and an immunoglobulin heavy chain constant region. In certain embodiments, fusions can be made at the amino terminus of the vibe protein or at the C-terminus of the vibe protein. In certain embodiments, the immunoglobulin heavy chain constant region is an Fc region.

The term “Fc” refers to a molecule or sequence including the sequence of a non-antigen-binding portion of antibody, whether in monomeric or multimeric form. The original immunoglobulin source of an Fc can be of human origin and can be from any isotype, e.g., IgG, IgA, IgM, IgE or IgD. One method of preparation of an isolated Fc molecule involves digestion of an antibody with papain to separate antigen and non-antigen binding portions of the antibody. Another method of preparation of isolated Fc molecules is production by recombinant DNA expression followed by purification of the Fc molecules so expressed. A full-length Fc consists of the following Ig heavy chain regions: CH1, CH2 and CH3 wherein the CH1 and CH2 regions are typically connected by a flexible hinge region. In certain embodiments, an Fc has an amino acid sequence of IgG1, for example, DKTHTCPPCPAPELLGGPSVFLFPPKPKDTLMISRTPE VTCVVVDVSHEDPEVKFNWYVDGVEVHNAKTKPREEQYNSTYRVVSVLTVLH QDWLNGKEYKCKVSNKALPAPIEKTISKAKGQPREPQVYTLPPSREEMTKNQVS LTCLVKGFYPSDIAVEWESNGQPENNYKTTPPVLDSDGSFFLYSKLTVDKSRWQ QGNVFSCSVMHEALHNHYTQKSLSLSPGK*. (SEQ ID NO:7). The terms “Fc protein, “Fc sequence”, “Fc molecule”, “Fc region” and “Fc portion” are taken to have the same meaning as “Fc”.

The term “Fc fragment” when used in association with Fc molecule, or fusion polypeptides thereof, refers to a peptide or polypeptide that includes less than the full length amino acid sequence of an Fc molecule. Such a fragment can arise, for example, from a truncation at the amino terminus, a truncation at the carboxy terminus, and/or an internal deletion of a residue(s) from the amino acid sequence. Fc fragments can result from alternative RNA splicing or from in vivo protease activity.

The term “Fc variant” when used in association with an Fc molecule, or with fusion polypeptides thereof, refers to a polypeptide including an amino acid sequence which contain one or more amino acid sequence substitutions, deletions, and/or additions as compared to native Fc amino acid sequences. Variants can be naturally occurring or artificially constructed. Variants of the presently disclosed subject matter can be prepared from the corresponding nucleic acid molecules encoding said variants, which have a DNA sequence that varies accordingly from the DNA sequences for native Fc molecule.

The term “derivative” when used in association with an Fc molecule, or with fusion polypeptides thereof, refers to Fc variants or fragments thereof, that have been chemically modified, as for example, by covalent attachment of one or more polymers, including, but limited to, water soluble polymers, N-linked or O-linked carbohydrates, sugars, phosphates, and/or other such molecules. The derivatives are modified in a manner that is different from native Fc, either in the type or location of the molecules attached to the polypeptide. Derivatives further include deletion of one or more chemical groups naturally attached to an Fc molecule.

The term “fusion” refers to joining of different peptide or protein segments by genetic or chemical methods wherein the joined ends of the peptide or protein segments can be directly adjacent to each other or can be separated by linker or spacer moieties such as amino acid residues or other linking groups.

An Fc, or a variant, fragment or derivative thereof, can be from an Ig class. In one embodiment, an Fc is from the IgG class, such as IgG1, IgG2, IgG3, and IgG4. In another embodiment, an Fc is from IgG1. An Fc can also include amino acid residues represented by a combination of any two or more of the Ig classes, such as residues from IgG1 and IgG2, or from IgG1, IgG2 and IgG3, and so forth.

In addition to naturally occurring variations in Fc regions, Fc variants, fragments and derivatives can contain non-naturally occurring changes in Fc which are constructed by, for example, introducing substitutions, additions, insertions or deletions of residues or sequences in a native or naturally occurring Fc, or by modifying the Fc portion by chemical modification and the like. In general, Fc variants, fragments and derivatives are prepared such that the increased circulating half-life of Fc fusions to RSpo1 is largely retained.

Also provided by the presently disclosed subject matter are Fc variants with conservative amino acid substitutions. Non-limiting examples of conservative amino acid substitutions are set forth hereinabove, and are also exemplified by substitution of non-naturally occurring amino acid residues which are typically incorporated by chemical peptide synthesis rather than by synthesis in biological systems. These include peptidomimetics, and other reversed or inverted forms of amino acid moieties. Conservative modifications to the amino acid sequence of an Fc region (and the corresponding modifications to the encoding nucleotides) are expected to produce Fc molecules (and fusion proteins including vibe proteins and Fc regions) which have functional and chemical characteristics similar to those of unmodified Fc molecules and fusion proteins including unmodified Fc regions.

In addition to the substitutions set forth above, any native residue in an Fc molecule (or in an Fc region of a fusion protein including a vibe protein) can also be substituted with alanine (Cunningham et al. Science 244, 1081-1085 (1989)).

Examples of Fc variants are disclosed in WO96/32478 and WO97/34630 hereby incorporated by reference. Furthermore, alterations can be in the form of altered amino acids, such as peptidomimetics or D-amino acids.

The Fc protein can also be linked to an RSpo protein by “linker” moieties including chemical groups or amino acids of varying lengths. Such chemical linkers are well known in the art. Amino acid linker sequences can include but are not limited to: (a) ala-ala-ala; (b) ala-ala-ala-ala; (c) ala-ala-ala-ala-ala; (d) gly-gly; (e) gly-gly-gly; (f) gly-gly-gly-gly-gly; (g) gly-gly-gly-gly-gly-gly-gly; (h) gly-pro-gly; (i) gly-gly-pro-gly-gly; and (j) any combination of subparts (a) through (i).

While Fc molecules can be used as components of fusion proteins with an RSpo protein, it is also contemplated that other amino acid sequences which bind to an FcRn receptor and confer increased in vivo half-life can also be used. Examples of such alternative molecules are described in U.S. Pat. No. 5,739,277, which is hereby incorporated by reference.

Vibe Nucleic Acid Agonists

The term “vibe nucleic acid” refers to isolated, non-naturally occurring nucleic acid encoding a vibe protein. For example, but not by way of limitation, a vibe nucleic acid can encode RSpo1. The term “RSpo1 nucleic acid” or “RSpo1” refers to isolated, non-naturally occurring nucleic acid encoding an RSpo1 protein, such as, but not limited to, nucleic acids having SEQ ID NO:1 (Genbank accession number DQ318235), fragments thereof, complement thereof, and derivatives thereof. An RSpo nucleic acid can also include Rspondin-2, which has an exemplary nucleotide sequence set forth as SEQ ID NO:3 (Genbank NM_{—}178565), and Rspondin-4, which has an exemplary nucleotide sequence set forth as SEQ ID NO:5 (Genbank accession number NM_{—}001029871). It is to be understood that the RSpo1, RSpo2, and RSpo4 nucleic acid sequences set forth above are exemplary vibe nucleic acid sequences. The disclosure expressly includes all fragments, variants and isoforms of these and other vibe nucleic acid sequences.

A non-naturally occurring nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See F. Ausubel, et al., ed. (1987) Current Protocols in Molecular Biology, Greene Publishing and Wiley Interscience, New York. In certain embodiments, a non-naturally-occurring nucleic acid of the present disclosure can be, for example, DNA or RNA and may or may not contain intronic sequences. In certain embodiments, the non-naturally-occurring nucleic acid of the present disclosure is a cDNA molecule.

The presently disclosed subject matter further provides for the use of variant RSpo polynucleotides, and fragments thereof, that differ from the nucleotide sequence shown in SEQ ID NO: 1, 3, and 5 due to degeneracy of the genetic code and thus encode the same protein as that encoded by the nucleotide sequence shown in SEQ ID NO: 1, 3, and 5.

The present disclosure also provides for the use of vibe nucleic acids, such as RSpo isolated, non-naturally occurring nucleic acid molecules encoding the variant polypeptides described above. Such polynucleotides can be as allelic variants (same locus), homologs (different locus), and orthologs (different organism) of the disclosed vibe nucleic acid sequences, or can be constructed by recombinant DNA methods or by chemical synthesis. Such variants can be made by mutagenesis techniques, including those applied to polynucleotides, cells, or organisms. Accordingly, as discussed herein, the variants can contain nucleotide substitutions, deletions, inversions and insertions.

In certain embodiments, variants have a substantial identity with the nucleic acid molecules of SEQ ID NO:1, 3, or 5 and the complements thereof. Variation can occur in either or both the coding and non-coding regions. The variations can produce both conservative and non-conservative amino acid substitutions.

Orthologs, homologs, and allelic variants can be identified using methods well known in the art. These variants include a nucleotide sequence encoding a polypeptide that is at least about 60-65%, 65-70%, 70-75%, 80-85%, 90-95% or more homologous to the nucleotide sequence shown in SEQ ID NO:1, 3, or 5 or a fragment of this sequence. In certain embodiments, the variant includes a nucleotide sequence encoding a polypeptide that is at least about 90-95% or more homologous to the nucleotide sequence shown in SEQ ID NO:1, 3, or 5 or a fragment of this sequence. Such nucleic acid molecules can readily be identified as being able to hybridize under stringent conditions, to the nucleotide sequence shown in SEQ ID NO:1, 3, or 5 or a fragment of the sequence.

In certain embodiments, nucleic acids including sequences encoding vibe proteins are administered to treat or prevent osteoporosis or bone-related disorders, by way of gene therapy. Gene therapy refers to therapy performed by the administration to a subject of an expressed or expressible nucleic acid. In this embodiment of the presently disclosed subject matter, the nucleic acids produce their encoded protein that mediates a therapeutic effect.

Any of the methods for gene therapy available in the art can be used according to the present disclosure. Exemplary methods are described below. For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In certain embodiments, the compound includes nucleic acid sequences encoding an RSpo polypeptide or functional fragment thereof, said nucleic acid sequences being part of expression vectors that express the vibe polypeptide or functional fragments thereof in a suitable host. In particular, such nucleic acid sequences have promoters operably linked to the RSpo coding region, said promoter being inducible or constitutive, and, optionally, tissue-specific.

Delivery of nucleic acid into a subject or cell can be either direct, in which case the subject or cell is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In certain embodiments, the nucleic acid can be directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, microcapsules, nanoparticles, or nanocapsules, or by administering them in linkage to a peptide which is known to enter the nucleus, by administering it in linkage to a ligand subject to receptor-mediated endocytosis (see, e.g., Wu and Wu, J. Biol. Chem. (1987); 262:4429-4432) (which can be used to target cell types specifically expressing the receptors), etc. The nucleic acid-ligand complexes can also be formed in which the ligand includes a fusogenic viral peptide to disrupt endosomes, allowing the nucleic acid to avoid lysosomal degradation. In addition, the nucleic acid can be targeted in vivo for cell specific uptake and expression, by targeting a specific receptor (see, e.g., PCT Publications WO 92/06180; WO 92/22635; WO92/20316; WO93/14188, WO 93/20221).

In certain embodiments, the nucleic acid can be introduced intracellularly and incorporated within host cell DNA for expression, by homologous recombination (Koller and Smithies, Proc. Natl. Acad. Sci. USA (1989); 86:8932-8935; ZijIstra et al., Nature (1989); 342:435-438).

In certain embodiments, a viral vector that contains nucleic acid encoding an RSpo polypeptide or a functional fragment thereof can be used. For example, a retroviral vector can be used (see Miller et al., Meth. Enzymol. (1993); 217:581-599). These retroviral vectors contain the components necessary for the correct packaging of the viral genome and integration into the host cell DNA. More detail about retroviral vectors can be found in Boesen et al., Biotherapy (1994); 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., J. Clin. Invest. (1994); 93:644-651; Kiem et al., Blood (1994); 83:1467-1473 ; Salmons and Gunzberg, Human Gene Therapy (1993); 4:129-141; and Grossman and Wilson, Curr. Opin. in Genetics and Devel. (1993); 3:110-114.

Adenoviruses are attractive vehicles for delivering genes. Adenoviruses naturally infect respiratory epithelia where they cause a mild disease. Other targets for adenovirus-based based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In certain embodiments, adenovirus vectors are used.

Adeno-associated virus (AAV) can also be used (Walsh et al., Proc. Soc. Exp. Biol. Med. 204:289-300 (1993); U.S. Pat. No. 5,436,146). Vectors that can be used in gene therapy are discussed below in detail below.

Another approach to gene therapy involves transferring a gene to cells in tissue culture by such methods as electroporation, lipofection, calcium phosphate mediated transfection, or viral infection, The method of transfer can further include the transfer of a selectable marker to the cells. The cells are then placed under selection to isolate those cells that have taken up and are expressing the transferred gene. Those cells are then delivered to a patient.

The nucleic acid can be introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and can be used in accordance with the presently disclosed subject matter, provided that the necessary developmental and physiological functions of the recipient cells are not disrupted. The technique should provide for the stable transfer of the nucleic acid to the cell, so that the nucleic acid is expressible by the cell and can be heritable and expressible by its cell progeny.

The resulting recombinant cells can be delivered to a patient by various methods known in the art. In certain embodiments, recombinant blood cells (e.g., hematopoietic stem or progenitor cells) are administered intravenously. The amount of cells envisioned for use depends on the desired effect, patient state, etc., and can be determined by one skilled in the art.

Cells into which a nucleic acid can be introduced for purposes of gene therapy encompass any desired, available cell type. Recombinant cells can also be used in gene therapy, where nucleic acid sequences encoding an RSpo protein or functional fragment thereof, are introduced into the cells such that they are expressible by the cells or their progeny, and the recombinant cells are then administered in vivo for therapeutic effect. For example, stem or progenitor cells can be used. Any stem and/or progenitor cells which can be isolated and maintained in vitro can potentially be used (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

### Vectors

The terms “vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below. A “therapeutic vector” as used herein refers to a vector which is acceptable for administration to a subject. A subject may be human or a non-human subject. Non-limiting examples of non-human subjects include non-human primates, dogs, cats, mice, rats, guinea pigs, rabbits, pigs, fowl, horses, cows, goats, sheep, cetaceans, etc.

Vectors typically include the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA can be from the same gene or from different genes, and can be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET plasmids (Invitrogen, San Diego, Calif.), pCDNA3 plasmids (Invitrogen), pREP plasmids (Invitrogen), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

Suitable vectors include, but are not limited to, viruses, such as adenoviruses, adeno-associated virus (AAV), vaccinia, herpesviruses, baculoviruses and retroviruses, parvovirus, lentivirus, bacteriophages, cosmids, plasmids, fungal vectors, naked DNA, DNA lipid complexes, and other recombination vehicles typically used in the art which have been described for expression in a variety of eukaryotic and prokaryotic hosts, and can be used for gene therapy as well as for simple protein expression.

Viral vectors, such as adenoviral vectors, can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). AAV vectors, such as those disclosed in U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see McConnell et al., 2004, Hum Gene Ther. 15(10:1022-33; Mccarty et al., 2004, Annu Rev Genet. 38:819-45; Mah et al., 2002, Clin. Pharmacokinet. 41(12):901-11; Scott et al., 2002, Neuromuscul. Disord. 12(Suppl 1):S23-9. In addition, see U.S. Pat. No. 5,670,488. Beck et al., 2004, Curr Gene Ther. 4(4): 457-67, specifically describe gene therapy in cardiovascular cells.

### Pharmaceutical Compositions

The presently disclosed subject matter also provides for pharmaceutical compositions which include at least one vibe protein, vibe gene, or functional fragment thereof, alone or in combination with at least one other agent, as described below. The presently disclosed subject matter further provides pharmaceutical compositions which include an RSpo agonist, e.g., all or portions of RSpo1 polynucleotide sequences, RSpo1 polypeptides or functional fragments thereof, or other RSpo1 agonists, alone or in combination with at least one other agent, such as a stabilizing compound, and can be administered in any sterile, biocompatible pharmaceutical carrier, including, but not limited to, saline, buffered saline, dextrose, and water. In certain embodiments, the composition can be in a liquid or lyophilized form and comprises a diluent (Tris, citrate, acetate or phosphate buffers) having various pH values and ionic strengths, solubilizer such as TWEEN™ or Polysorbate, carriers such as human serum albumin or gelatin, preservatives such as thimerosal, parabens, benzylalconium chloride or benzyl alcohol, antioxidants such as ascrobic acid or sodium metabisulfite, and other components such as lysine or glycine. Selection of a particular composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980).

The methods of the presently disclosed subject matter find use in treating or preventing osteoporosis or bone-related disorders. Peptides can be administered to the patient intravenously and/or subcutaneously in a pharmaceutically acceptable carrier such as physiological saline. In certain embodiments, the carrier can be a solvent or dispersion medium containing, for example, water, ethanol, polyol (for example, glycerol, propylene glycol, and liquid polyetheylene glycol, and the like), and suitable mixtures thereof. Standard methods for intracellular delivery of peptides can be used (e.g., delivery via liposome). Such methods are well known to those of ordinary skill in the art. The formulations of this presently disclosed subject matter are useful for parenteral administration, such as intravenous, subcutaneous, intramuscular, and intraperitoneal. Therapeutic administration of a polypeptide intracellularly can also be accomplished using gene therapy. The route of administration eventually chosen will depend upon a number of factors and can be ascertained by one skilled in the art.

In certain embodiments, the pharmaceutical compositions of the presently disclosed subject matter can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated. Pharmaceutically compatible binding agents, and/or adjuvant materials can be included as part of the composition. The tablets, pills, capsules, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

In certain embodiments, pharmaceutical compositions of the presently disclosed subject matter can also be prepared wherein the RSpo agonist of the disclosure is covalently or non-covalently attached to a nanoparticle. By way of example, but not limitation, a nanoparticle can be a dendrimer, such as the polyamidoamine employed in Kukowska-Latallo et al., (2005) Cancer Res., vol. 65, pp. 5317-24, which is incorporated herein by reference in its entirety. Other dendrimers that can be used in conjunction with the RSpo agonists of the instant disclosure include, but are not limited to, polypropylenimine dendrimers as described in U.S. Pat. No. 7078461, which is hereby incorporated by reference in its entirety.

Pharmaceutical compositions suitable for use in the present disclosure include compositions wherein the active ingredients are contained in an effective amount to achieve the intended purpose. As used herein, the term “therapeutically effective amount” refers to an amount of an RSpo agonist or other vibe agonist which promotes bone mineral density (BMD) or bone mineral concentration (BMC) to a target BMD or BMC, or to a target BMD or BMC range that provides benefit to a patient or, alternatively, maintains a patient at a target BMD or BMC, or within a target BMD or BMC range. Alternatively, the term “therapeutically effective amount” refers to an amount of an RSpo agonist or other vibe agonist which promotes bone growth. The amount will vary from one individual to another and will depend upon a number of factors, including the overall physical condition of the patient, severity and the underlying cause of osteoporosis or bone-related disorder. It is understood that such targets will vary from one individual to another such that physician discretion may be appropriate in determining an actual target BMD or BMC for any given patient. Nonetheless, determining a target BMD or BMC is well within the level of skill in the art.

The pharmaceutical formulations of the presently disclosed subject matter can be administered for prophylactic and/or therapeutic treatments. For example, in certain embodiments, pharmaceutical compositions of the presently disclosed subject matter are administered in an amount sufficient to treat, prevent and/or ameliorate osteoporosis or a bone-related disorder. As is well known in the medical arts, dosages for any one patient depends upon many factors, including stage of the disease or condition, the severity of the disease or condition, the patient's size, body surface area, age, the particular compound to be administered, sex, time and route of administration, general health, and interaction with other drugs being concurrently administered.

Accordingly, in certain embodiments of the presently disclosed subject matter, vibe nucleotide and amino acid sequences, such as, but not limited to, RSpo nucleotide and RSpo amino acid sequences, can be administered to a patient alone, or in combination with other nucleotide sequences, drugs or hormones, or other agents used in the treatment or prevention of osteoporosis or bone-related disorders or symptoms thereof as described herein or known in the art, or in pharmaceutical compositions where it is mixed with excipient(s) or other pharmaceutically acceptable carriers. In certain embodiments, the pharmaceutically acceptable carrier is pharmaceutically inert. In certain embodiments, vibe polynucleotide sequences or vibe amino acid sequences can be administered alone to individuals subject to or suffering from osteoporosis or a bone-related disorder. In certain embodiments, vibe nucleotide and amino acid sequences can be administered alone to individuals subject to or suffering from osteoporosis or a bone-related disorder. The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Ear. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels.

Single or multiple administrations of formulations can be given depending on the dosage and frequency as required and tolerated by the patient. In certain embodiments, the formulations will provide a sufficient quantity of active agent to effectively treat, prevent or ameliorate osteoporosis or a bone-related disorder or symptoms thereof as described herein. In certain embodiments, the vibe agonists of the presently disclosed subject matter are administered once, twice, or three, four, five, or six times per week, or daily. In certain embodiments, RSpo proteins or genes of the presently disclosed subject matter are administered once, twice, or three, four, five, or six times per week, or daily. In certain embodiments, the vibe agonists of the presently disclosed subject matter can be administered one or more times per day. In certain embodiments, the RSpo proteins or genes of the presently disclosed subject matter are administered one or more times per day. For example, but not by way of limitation, an exemplary pharmaceutical formulation for oral administration, intravenous injection or subcutaneous injection can be in a daily amount of between about 0.1 to 0.5 to about 20, 50, 100, 1000, 3000 or 4000 or more μg per kilogram of body weight per day of protein. In certain embodiments, dosages are from about 1 mg to about 4 mg per kg of body weight per patient per day of protein are used. For example, in certain embodiments a therapeutically effective amount of a polypeptide of the presently disclosed subject matter is a dosage of between about 0.025 to 0.5 milligram per 1 kilogram of body weight of the patient; or, a therapeutically effective amount is a dosage of between about 0.025 to 0.2 milligram, or 0.05 to 0.1 milligram, or 0.075 to 0.5 milligram, or 0.2 to 0.4 milligram, of the compound per 1 kilogram of body weight of the patient. In certain embodiments, a therapeutically effective amount of a polypeptide of the presently disclosed subject matter is a dosage of between about 0.02 to about 5 milligram of the polypeptide per 1 kilogram of body weight of the patient. For example, a therapeutically effective amount of a polypeptide of this disclosure is a dosage of between about 3 to 4 milligram of the polypeptide per 1 kilogram of body weight of the patient.

Toxicity and therapeutic efficacy of such compounds can be determined by standard pharmaceutical procedures in cell cultures or experimental animals (LD50, the dose lethal to 50% of the population; and ED50, the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. In certain embodiments, the dosage of such compounds lies within a range of circulating concentrations that include the ED50 with little or no toxicity. The dosage varies within this range depending upon the dosage form employed, sensitivity of the patient, and the route of administration.

### Diagnosis of Osteoporosis and Bone-Related Disorders

A variety of methods can be employed for the diagnostic and prognostic evaluation of bone loss, osteoporosis and bone-related disorders, and for the identification of subjects having a predisposition or at risk for such disorders. Such methods can, for example, utilize reagents such as vibe (e.g., RSpo1) nucleotide sequences or vibe (e.g., RSpo1) antibodies. Specifically, such reagents can be used, for example, for the detection of either over- or under-expression of vibe (e.g., RSpo1) mRNA relative to the non-osteoporosis or non-bone-related disorder state or the detection of either an over- or an under-abundance of vibe (e.g., RSpo1) gene product relative to the non-osteporosis or non-bone-related disorder state.

In certain embodiments, the method for the diagnostic and prognostic evaluation of bone loss, osteoporosis and bone-related disorders, and for the identification of subjects having a predisposition or at risk for such disorders can utilize reagents for the detection of one or more RSpo genes or proteins, e.g., RSpo1. For example, such reagents can be used for the detection of either over- or under-expression of the mRNA of one or more RSpo genes relative to the non-osteoporosis or non-bone-related disorder state or the detection of either an over- or an under-abundance of one or more RSpo proteins relative to the non-osteoporosis or non-bone-related disorder state.

The methods described herein can be performed, for example, by utilizing pre-packaged diagnostic kits including at least one specific RSpo (e.g., RSpo1) nucleotide sequence or RSpo (e.g., RSpo1) antibody reagent described herein, which can be conveniently used, e.g., in clinical settings, to diagnose patients having bone mass abnormalities or experiencing bone loss. In certain embodiments, the methods described herein can be performed, for example, by utilizing pre-packaged diagnostic kits including at least one specific vibe nucleotide sequence or vibe antibody reagent.

For the detection of vibe gene expression or gene products, e.g., RSpo gene expression or RSpo gene products, any cell type or tissue in which an RSpo gene is expressed, can be utilized.

The level of vibe gene expression, e.g., RSpo gene expression, can be assayed by detecting and measuring vibe transcription. For example, RNA from a cell type or tissue known, or suspected, to express the vibe gene can be isolated and tested utilizing hybridization or PCR techniques as known in the art. The isolated cells can be obtained from cell culture or from a patient. In certain embodiments, the analysis of cells taken from culture may be a necessary step in the assessment of cells to be used as part of a cell-based gene therapy technique or, alternatively, to test the effect of compounds on the expression of an vibe gene. Such analyses can reveal both quantitative and qualitative aspects of the expression pattern of the vibe gene, including activation or inactivation of vibe gene expression.

In certain embodiments of such a detection scheme, eDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are produced based on methods known in the art. In certain embodiments, the lengths of such nucleic acid reagents are at least 9-30 nucleotides. For detection of the amplified product, the nucleic acid amplification can be performed using radioactively or non-radioactively labeled nucleotides. Alternatively, enough amplified product can be made such that the product can be visualized by standard ethidium bromide staining or by utilizing any other suitable nucleic acid staining method.

Additionally, it is possible to perform such vibe gene expression assays in situ, i.e., directly upon tissue sections (fixed and/or frozen) of patient tissue obtained from biopsies or resections, such that no nucleic acid purification is necessary. Nucleic acid reagents can be used as probes and/or primers for such in situ procedures (See, e.g., Nuovo, G. J., 1992, PCR In Situ Hybridization: Protocols And Applications, Raven Press, NY).

In certain embodiments, where a sufficient quantity of the appropriate cells can be obtained, standard Northern analysis can be performed to determine the level of mRNA expression of the vibe gene.

Antibodies directed against wild type or mutant vibe gene products or conserved variants or peptide fragments thereof, can also be used as osteporosis or bone-related disorder diagnostics and prognostics, as described herein. Such diagnostic methods, which can be used to detect abnormalities in the level of vibe gene expression, can be performed in vivo or in vitro.

Such antibodies can be labeled, for example, with a radio-opaque or other appropriate compound and injected into a subject in order to visualize binding to the RSpo expressed in the body using methods such as X-rays, CAT-scans, or MRI.

The tissue or cell type to be analyzed will generally include those which are known, or suspected, to express the vibe gene, e.g., mesenchymal progenitor cells (MPCs). The protein isolation methods employed herein can, for example, be such as those described in Harlow and Lane (Harlow, E. and Lane, D., 1988, Antibodies: A Laboratory Manual, Cold Spring Harbor Laboratory Press, Cold Spring Harbor, N.Y.), which is incorporated herein by reference in its entirety. The isolated cells can be derived from cell culture or from a patient. In certain embodiments, the analysis of cells taken from culture may be a necessary step in the assessment of cells that could be used as part of a cell-based gene therapy technique or, alternatively, to test the effect of compounds on the expression of the RSpo gene.

For example, antibodies, or fragments of antibodies, useful in the present disclosure can be used to quantitatively or qualitatively detect the presence of vibe gene products or conserved variants or peptide fragments thereof. This can be accomplished, for example, by immunofluorescence techniques employing a fluorescently labeled antibody (see below, this Section) coupled with light microscopic, flow cytometric, or fluorimetric detection.

The antibodies (or fragments thereof) or vibe fusion or conjugated proteins useful in the present disclosure can, additionally, be employed histologically, as in immunofluorescence, immunoelectron microscopy or non-immuno assays, for in situ detection of vibe gene products or conserved variants or peptide fragments thereof, or for vibe binding (in the case of labeled vibe fusion protein).

In situ detection can be accomplished by removing a histological specimen from a patient, and applying thereto a labeled antibody or fusion protein of the presently disclosed subject matter. The antibody (or fragment) or fusion protein can be applied by overlaying the labeled antibody (or fragment) onto a biological sample. Using the presently disclosed subject matter, those of ordinary skill will readily perceive that any of a wide variety of histological methods (such as staining procedures) can be modified in order to achieve such in situ detection.

Immunoassays and non-immunoassays for vibe gene products or conserved variants or peptide fragments thereof will typically include incubating a sample, such as a biological fluid, a tissue extract, freshly harvested cells, or lysates of cells which have been incubated in cell culture, in the presence of a detectably labeled antibody capable of identifying RSpo gene products or conserved variants or peptide fragments thereof, and detecting the bound antibody by any of a number of techniques well-known in the art.

The biological sample can be brought in contact with and immobilized onto a solid phase support or carrier such as nitrocellulose, or other solid support which is capable of immobilizing cells, cell particles, or soluble proteins. The support can then be washed with suitable buffers followed by treatment with the detectably labeled vibe antibody or vibe fusion protein. The solid phase support can then be washed with the buffer a second time to remove unbound antibody or fusion protein. The amount of bound label on solid support can then be detected by conventional means.

With respect to antibodies, one of the ways in which the vibe antibody can be detectably labeled is by linking the same to an enzyme and use in an enzyme immunoassay (EIA) (Voller, A., The Enzyme Linked Immunosorbent Assay (ELISA), 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme, which is bound to the antibody, will react with an appropriate substrate, such as a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect RSpo through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the presently disclosed subject matter. Bioluminescence is a type of chemiluminescence found in biological systems, in which a catalytic protein increases the efficiency of the chemiluminescent reaction. The presence of a bioluminescent protein is determined by detecting the presence of luminescence. Bioluminescent compounds for purposes of labeling include luciferin, luciferase, and aequorin.

### Screening Methods

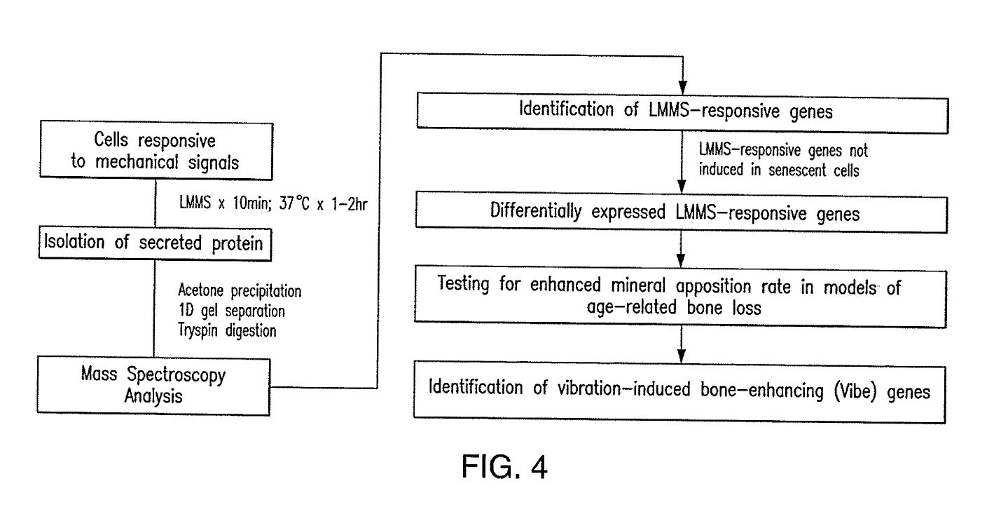

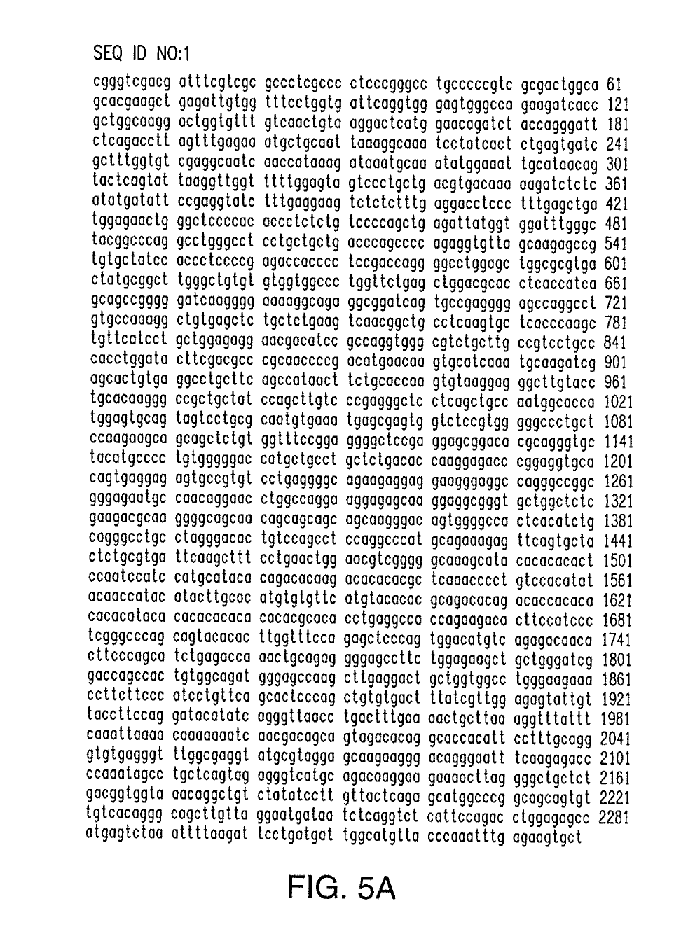

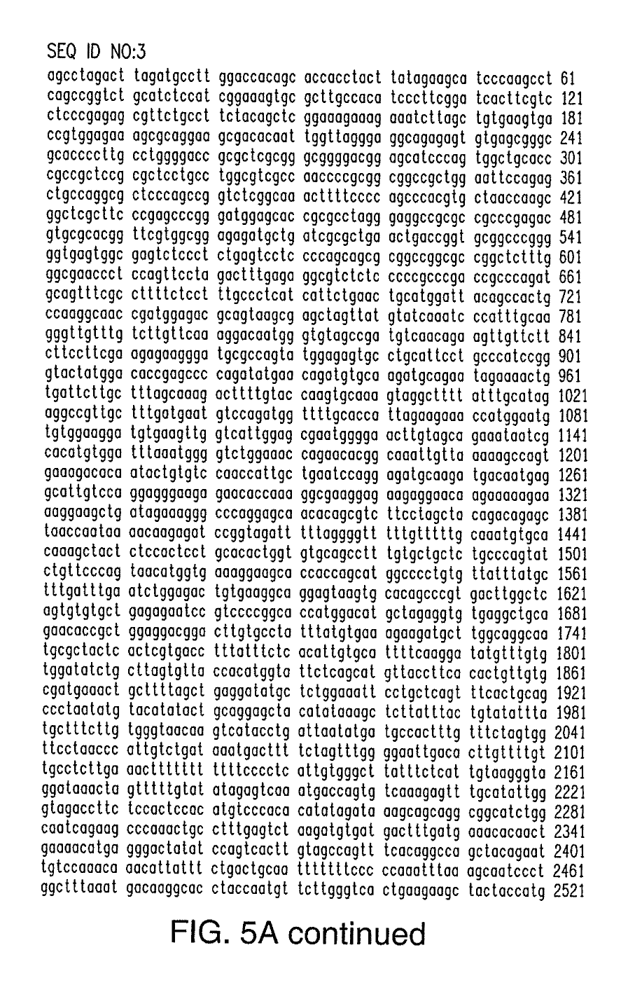

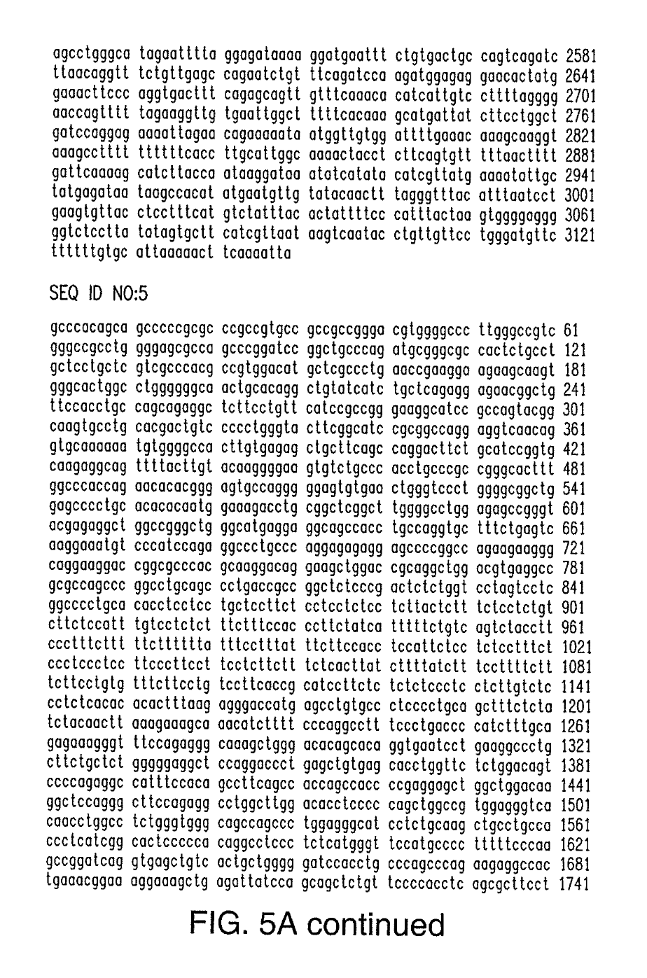

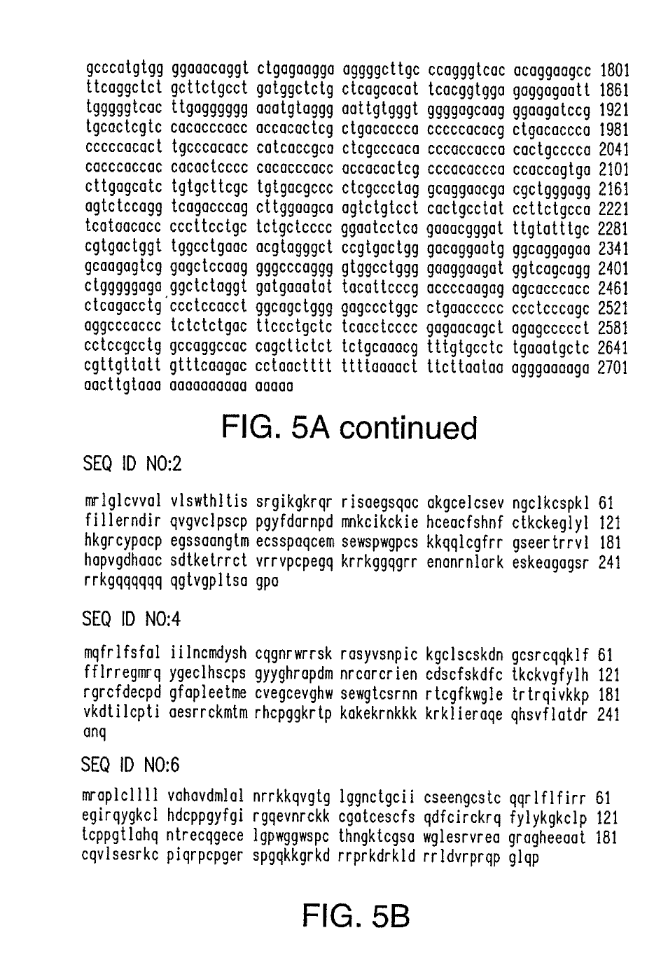

In another aspect, the disclosure provides screening methods for identifying additional proteins (e.g., secreted proteins or intracellular proteins) that are up- or down-regulated in response to stimuli including, but not limited to, mechanical stimuli, such as low magnitude mechanical signals (LMMS). These proteins that are upregulated can be referred to as vibration-induced proteins, a subset of which can be referred to as vibration-induced bone-enhancing (vibe) proteins. FIG. 4 outlines an exemplary screening method for the identification of secreted vibe proteins. This method is applicable to any mechanically-sensitive cell type, including MPCs. The method is also applicable to intracellular vibe proteins and is not limited to secreted proteins.

Proteins identified using the screening methods described herein are candidate targets for modulation of bone formation and use in preventing and treating osteoporosis and bone-related disorders. For example, proteins identified as up-regulated in response to stimuli, e.g., LMMS, are implicated as proteins that are involved in increasing bone formation. Proteins identified as down-regulated in response to LMMS are implicated as proteins that are involved in inhibiting bone formation.

In certain embodiments, cells, e.g, MPCs, are subjected to stimuli such as LMMS using any method known in the art. For example, cells can be subjected to vibration for a period of time, e.g, about 1-10 minutes or more, as described herein. In certain embodiments, the cells are subjected to vibration for a period of about 10 minutes, about 20 minutes, about 25 minutes, about 30 minutes, about 40 minutes, about 50 minutes, about 1 hour, or more. In certain embodiments, the vibration can be delivered at a sinusoidal frequency of about 32 to about 37 Hz, or at such frequency known or discovered to stimulate the cells. Following stimuli, in order to identify vibe genes, secreted or intracellular proteins can be precipitated from media or cell lysate and quantified by, for example, Western Blotting, HPLC, and/or mass spectroscopy analysis. Expression levels of the secreted or intracellular proteins subjected to simuli are then compared to proteins expressed by unstimulated controls to identify proteins that were up- or down-regulated in response to the stimuli.

In certain embodiments, a comparison of expression levels is carried out between proteins produced in aged cells and proteins produced in non-senescent cells, in response to such stimuli. In certain embodiments, proteins which are over- or under-expressed in the aged cells versus the non-senescent cells are identified as targets for modulation of bone formation.

Once proteins are identified as being up- or down-regulated in response to stimuli, e.g., LMMS, and optionally differentially between aged cells and non-senescent cells, the ability of the protein to induce (or inhibit) bone formation can be further characterized in vivo using an animal model for osteoporosis or a bone-related disorder, as described herein or known in the art. For example, a mutant mouse model with an accelerated osteoporotic phenotype, such as the Terc^{-/-} model, or physiologically aged mice. In one embodiment, mineral apposition rate can be measured to assess bone formation ability of the candidate proteins.

The following example is offered to more fully illustrate the presently disclosed subject matter, but is not to be construed as limiting the scope thereof.

### EXAMPLE

### Example 1

### R-Spondin 1 Promotes Vibration-Induced Bone Formation in Mouse Models of Osteoporosis

### Materials and Methods

### Animals

Fourth-generation 6 month old Terc^{-/-} and 3-month old Wrn^{-/-} Terc^{-/-} mice, ^{36,37,44} as well as 18.5 month old physiologically aged male mice (all on the C57B1/6 background) were used for experiments. The University of Pennsylvania Institutional Animal Care and Use Committee (IACUC) approved the use of mice described in this Example.

### MPC Cell Strains and Culture

Bone marrow was flushed from femoral head samples collected post-hip arthroplasty from elective procedures and suspended in α-Minimal Essential Medium (MEM) with nucleosides (Gibco/Invitrogen, Grand Island, N.Y., USA) plus 10% fetal bovine serum (FBS) (Gibco/Invitrogen, Grand Island, N.Y., USA). Marrow was spun out of suspension at 300×g for 5 minutes and the pellet resuspended in α-MEM +10% FBS before seeding cells into tissue culture flasks. Non-adherent cells were rinsed from the flasks after 24 hours, and MPC strains were established based on selection by plastic-adherence. MPC cultures were grown and maintained in α-MEM+10% FBS. Cells were seeded at a density of 1×10⁴ cells/cm² at each passage and grown until confluent, usually by 10 days. Cells were used at the third confluent in vitro passage, after no more than ˜6 population doublings after outgrowth from explant, and were considered “early passage. Cultures were defined as being at the end of their proliferative lifespan or “senescent” when they were unable to complete one population doubling during a 4-week period that included three consecutive weeks of refeeding with fresh medium containing 10% FBS. Population doublings were calculated as previously described.⁴⁸ Collection of femoral head samples was approved by the Institutional Review Board of the University of Pennsylvania.

### Delivery of Low Magnitude Mechanical Signals (LMMS)

The Juvent 1000 Dynamic Motion Platform was used to deliver low magnitude mechanical signals (LMMS)⁴⁵. Amplitude was delivered at a sinusoidal frequency of 32-37 Hz (displacement frequency), acceleration “g” force of 0.3 g (peak to peak) (+/−20%), and vertical displacement of ˜85 μm with a continuous duty cycle. Cultures and additional mass ≧15.8 kg were placed on the platform to obtain a normal operating load. MPCs, serum-starved for 24 hr in α-MEM, were stimulated by LMMS for 10 min at room temperature, and then incubated for an additional 1 or 2 hrs at 37° C. prior to collection of secreted proteins, whole cell lysate, and whole cell RNA. For vibratory stimulation in a 29 year-old male subject, the same device was used and LMMS were delivered for 20 minutes.

### Blood Collection and Isolation of Plasma

Blood was collected prior to and one hour after delivery of LMMS by a vibratory platform as described above. Whole blood was collected into VACUTAINER® ethylenediaminetetraacetic acid (EDTA)-treated specimen tubes (BD, Franklin Lakes, N.J.). Cells were removed from plasma by layering blood over Ficoll-Paque PLUS (GE Healthcare, Mickleton, N.J.) and centrifugation per the manufacturer's instructions.

### SDS-PAGE, In-Gel Trypsin Digestion, and Mass Spectroscopy

Secreted proteins were precipitated from serum-free conditioned media with acetone at a 5:1 ratio, spun down for 10 min at 15,000×g at −20° C., and the pellet dried for 30 min. The protein was dissolved in RIPA buffer (150 mM NaCl, 1.0% IGEPAL® CA-630, 0.5% sodium deoxycholate, 0.1% SDS, 50 mM Tris, pH 8.0.) with 1× proteinase inhibitor cocktail (Sigma, St. Louis, Mo., USA) and phosphatase inhibitor cocktail (Pierce, Rockford, Ill., USA) and quantified by the BCA assay (Pierce, Pierce, Rockford, Ill., USA). One-dimensional SDS-PAGE was performed using standard techniques. After Coomassie blue staining and washing of the gel, contiguous gel fragments were excised and each slice cut into 1×1 mm pieces. The in-gel tryptic digestion kit #89871 (Pierce Biotechnology, Rockford, Ill., USA) was used according to the manufacturer's instructions with both reduction and alkylation steps as described. Extracted proteins were dried and refrigerated until submission for mass spectroscopy analysis by the Proteomics Core at the University of Pennsylvania. Identification of genes and relative expression patterns (prior to Western blot confirmation) were performed using Scaffold 3.0 software (Proteome Software, Portland, Oreg., USA). Relative expression of secreted proteins was normalized to the average abundance of 4 secreted proteins that did not change significantly with LMMS after replicate analysis. Data was pooled from MPCs derived from 3 individuals (age range 40-50 years old).

### Western Blot Analyses

After removal of conditioned media, LMMS-stimulated MPCs and controls (no LMMS stimulation) were lysed in RIPA buffer with 1× proteinase inhibitor cocktail (Sigma, St Louis, Mo., USA) and phosphatase inhibitor cocktail (Pierce, Rockford, Ill., USA) to recover total protein. The lysate was incubated on ice for 15 min and centrifuged at 12,000 g for 10 min. Protein in the supernatant was quantified using the BCA Assay kit (Pierce, Rockford, Ill., USA). Secreted protein from conditioned media was prepared as described for SDS-PAGE. Fifty micrograms of total protein was separated on a 10% SDS-PAGE gel and then transferred to PVDF membranes (Bio-Rad, Hercules, Calif., USA) by electroblotting. Membranes were incubated overnight at 4° C. with a 1:1000 dilution of antibody specific for Rspo1 (ab73760; ABCAM, Cambridge, Mass., USA). Membranes were washed with PBST (Phosphate buffered saline+0.1% Tween-20) and incubated for 1 hr with a 1:5000 goat anti-rabbit IgG-HRP (se-2004; Santa Cruz Biotechnology, Santa Cruz, Calif., USA) Antibodies against albumin (Cell Signaling, Danvers, Mass.), against β-actin, (Santa Cruz Biotech, Dallas, Tex.) and type I collagen (ABCAM, Cambridge, Mass.) were used at dilutions of 1:2000, 1:1000, and 1:1000, respectively. Appropriate anti-mouse or anti-rabbit HRP-conjugated secondary antibodies (Santa Cruz biotech, Dallas, Tex.) were used at a dilution of 1:5000.

### RNA Extraction and Real-Time PCR

Whole cell RNA was isolated at the same time as protein extraction from duplicate cultures using the RNeasy mini-kit (Qiagen, Valencia, Calif., USA). Real-time PCR was performed by standard methods.

### Preparation and Administration of Purified Rspo1

Human recombinant Rspo1 was prepared by the Wistar Institute Protein Expression Laboratory as previously described.⁴⁶ Animals were administered Rspo1 at 3.3 μg/g body weight as daily peritoneal injections for seven days.

### Mineral Apposition Rate

Bone histomorphometry was carried out essentially as previously described.⁴⁹ To determine mineral apposition rate (MAR), each animal was injected intraperitoneally with 30 mg/kg calcein (Sigma, St Louis, Mo., USA) at 9 days and 2 days before necropsy. Mouse hind limbs were excised, cleaned of soft tissue, and fixed in 3.7% formaldehyde for 72 hours. Isolated bone tissue was dehydrated in graded alcohols (70 to 100%), cleared in xylene and embedded in methyl methacrylate. Plastic tissue blocks were cut into 5 μm sections using a Polycut-S motorized microtome (Reichert-Jung, Nossloch, Germany). For TRAP staining, sections were incubated with substrate solution (112 mM sodium acetate, 77 mM L-(+) tartaric acid, 0.3% glacial acetic acid) at 370 C for 5 hrs. This solution was then replaced with substrate solution plus 11.6 mM sodium nitrite and 2.6 mM pararosaniline dye, and incubated at room temperature for an additional 2 hrs before rising and dehydration by standard procedures. Goldner's Trichrome staining, was performed by standard methods.

Three consecutive sections per limb were visualized for fluorochrome labeling using a Nikon Eclipse 90i microscope and Nikon Plan Fluor 10× objective (Nikon Inc., Melville, N.Y., USA). MAR was calculated by measuring the distance between the two resulting calcein fronts in bone sections. For other measurements, consecutive sections were visualized using 4× and 20× objectives. Image capture was performed using NIS Elements Imaging Software 3.10 Sp2 and a Photometrics Coolsnap EZ camera. The Bioquant Osteo II digitizing system (R&M Biometrics, Nashville, Tenn.) was used according to the manufacturer's instructions. Measurements for mineral apposition rate (MAR) were collected from the distal end of the femur at 100× magnification. The terminology and calculations used are those recommended by the Histomorphometry Nomenclature Committee of the ASBMR.⁴⁷

### Statistical Methods

The t test (Student's t test; two-sided and paired) was used to determine whether the average value for a bone histomorphometric parameter differed significantly between Rspo1-treated and Rspo1-untreated animals. One-way ANOVA was performed to determine if mRNA expression of Rspo1, Rspo2, and Rspo4 varied significantly with time after LMMS. Statistical significance was set to p=0.05. Statistical analysis was performed using GraphPad Prism4.0 (San Diego, Calif.). Error is expressed as standard error of the mean.

### Results

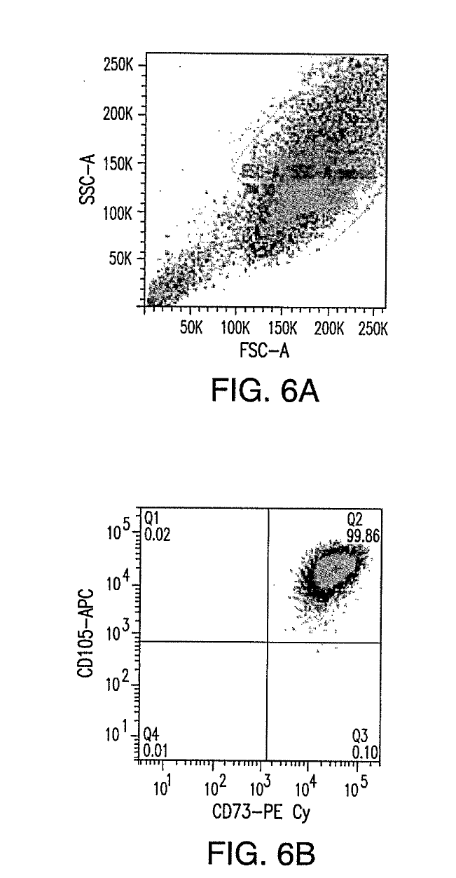

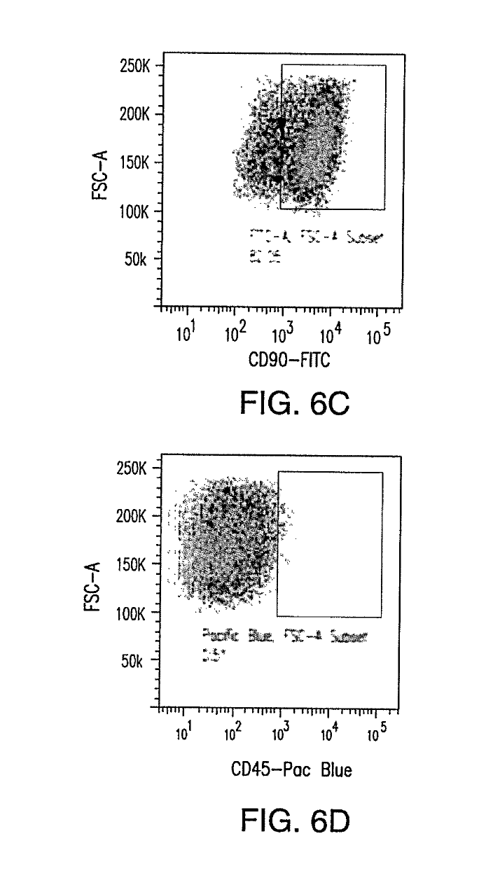

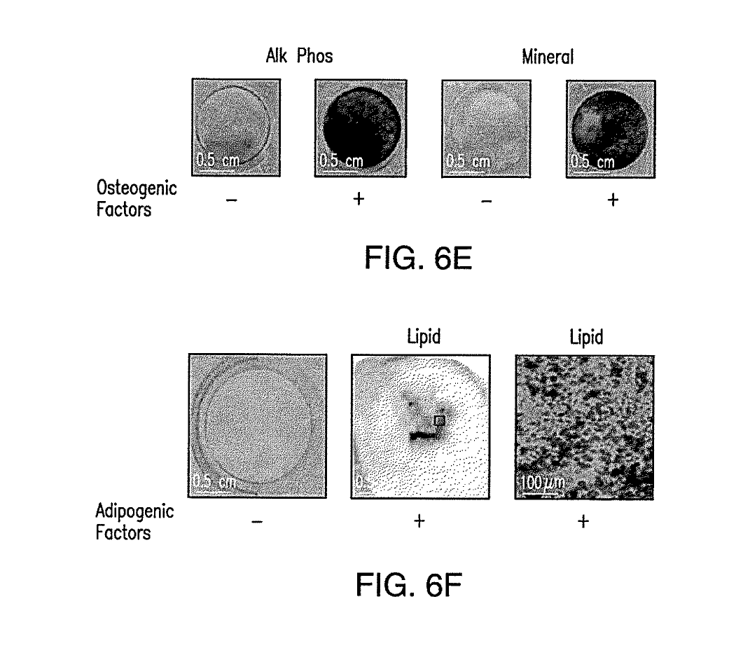

MPCs, which are among the mechanosensitive cells that reside in bone tissue, were used in this Example. Specifically, human CD73+ CD90+ CD105+ CD45-MPCs capable of differentiation into osteoblasts and adipocytes were used (FIG. 6). In order to identify secretory vibration-induced bone-enhancing (vibe) genes in human MPCs, proteins were precipitated from conditioned media 2 hours after LMMS and separated by 1-dimensional SDS-PAGE. After in-gel trypsin digestion of LMMS-enhanced Coomassie blue stained protein bands (centered at 75 kDa, 70 kDa, 45 kDa, and 30 kDa), HPLC, and mass spectroscopy analysis, 46 proteins were found to be up-regulated in response to LMMS compared to unstimulated controls. In response to LMMS, relative expression varied from about 2.5-to almost 30-fold.

The most highly expressed LMMS-induced proteins secreted by human MPCs are shown in Table 1, below. Of the known protein products, the species most highly up-regulated by LMMS was R-Spondin 1 (RSpo1), a Wnt pathway modulator with few reported effects in bone.¹⁹ Additional proteins identified as LMMS-responsive secretory proteins include Tissue inhibitor of metalloproteinases (TIMPs), Plasminogen activator inhibitor-1 (PAI-1) precursor, Collagen alpha 1 chain precursor variant, Fibrillin-1 precursor, and unidentified protein products, NCBI GI:62822120, GI:189053417, GI:189055325, G1:158258302, and G1:158256710 (Table 1).

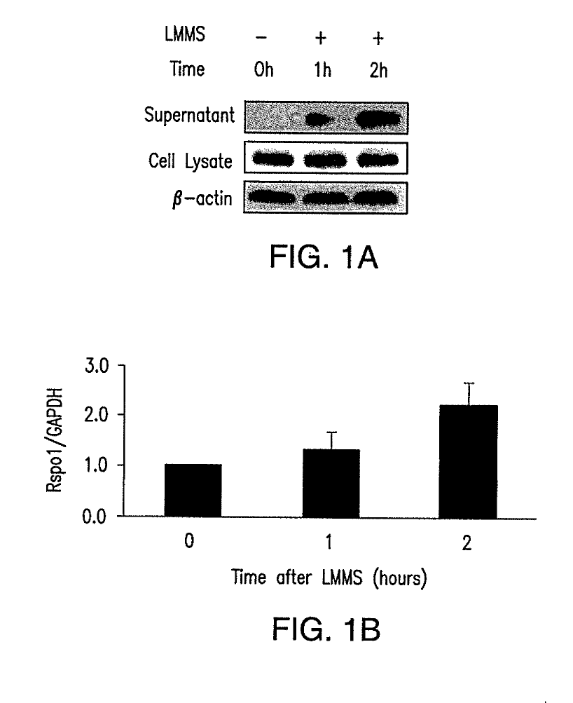

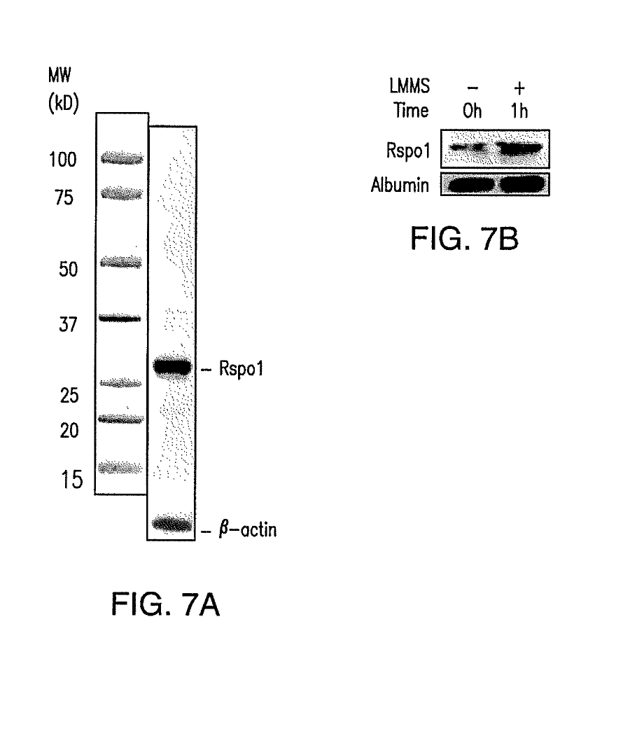

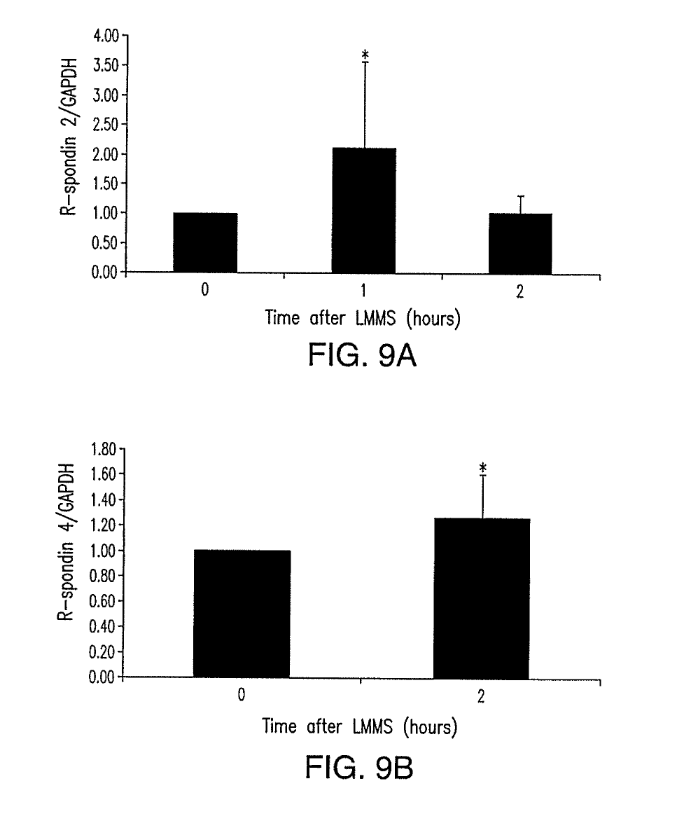

Rspo1 is recognized as a protein of approximately 30 kD by Western Blot analysis of whole cell protein (FIG. 7A). The vibration-enhanced expression of RSpo1 in human MPCs was confirmed by both Western blot analysis and RT-PCR (FIG. 1). An increase in the circulating level of RSpo1 was observed in response to LMMS in a 29-year old healthy male (FIG. 7B). In addition, in MPCs, mRNA levels of R-Spondin 2 and R-Spondin 4 increased by approximately 100% and 50%, respectively, in response to vibration stimulation (FIG. 9A-B). Success in finding that both known and unknown secretory proteins are responsive to LMMS, as well as the identification of genes that play known or suspected roles in bone or matrix remodeling, indicated that a subset of vibration-induced genes have the capacity to promote bone formation in vivo, and thus are true vibe genes.

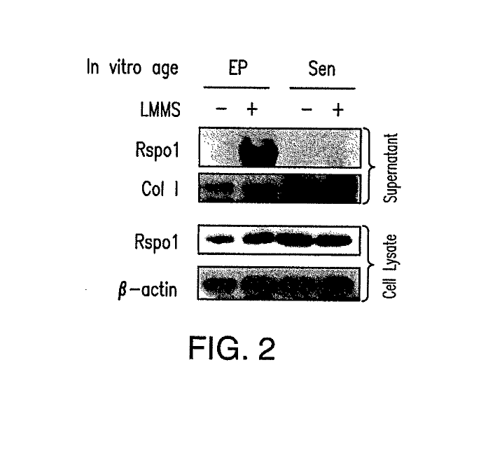

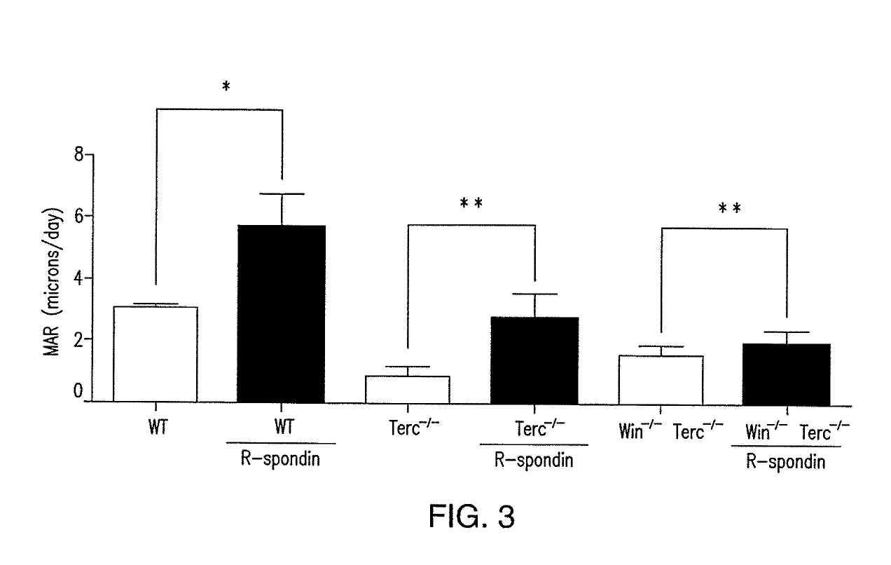

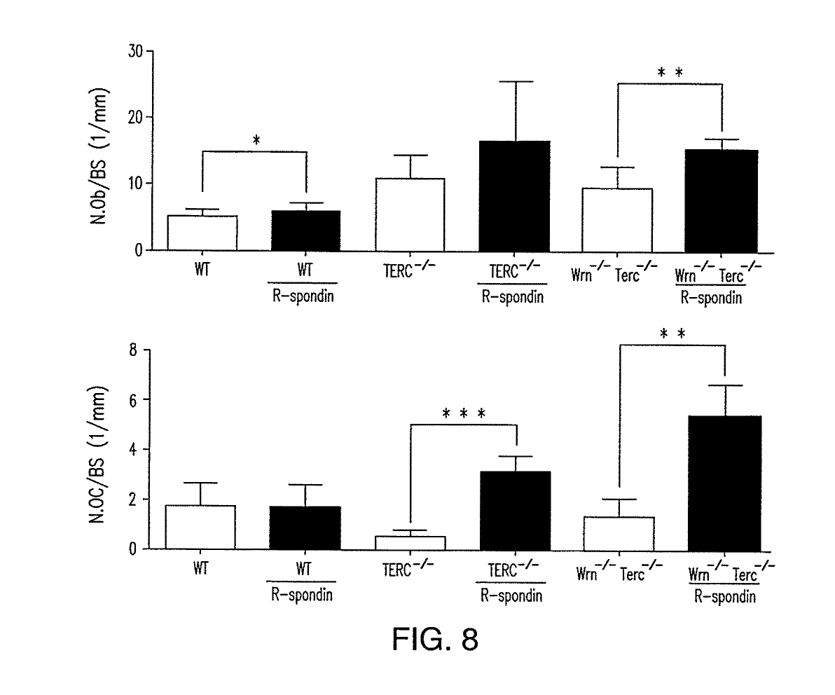

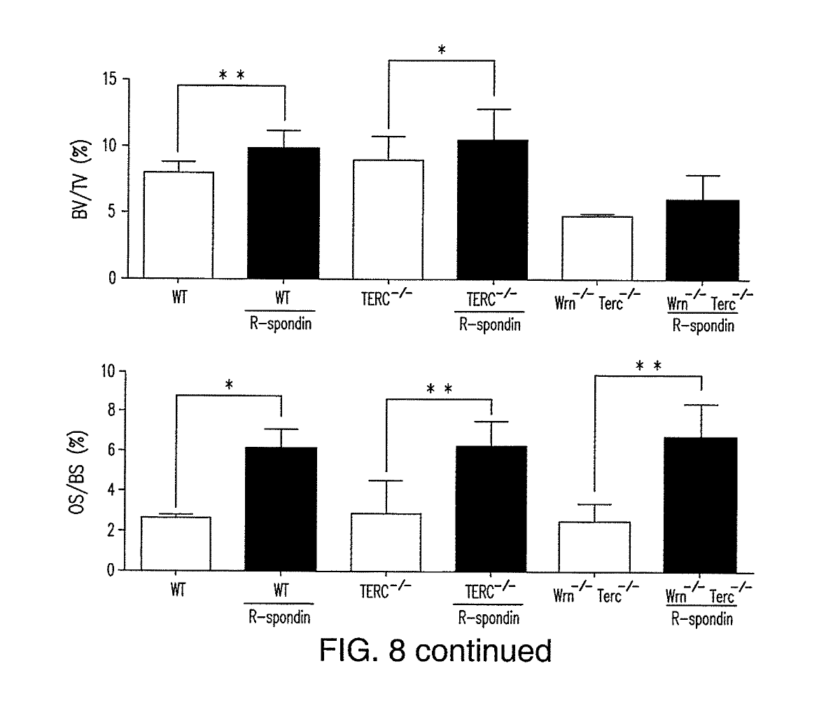

Rspo1 was chosen as a likely vibe gene based on its enhanced induction by LMMS (Table 1, FIG. 1), potential relevance to bone remodeling based on its characterization as a Wnt pathway modulator,^{19, 20} and its diminished secretion in senescent MPCs (FIG. 2). Its ability to increase mineral apposition in vivo was evaluated using three mouse models of age-bone-related loss. FIG. 3 shows that mineral apposition rate (MAR) is significantly increased when recombinant Rspo1 is administered to physiologically aged mice, as well as in two telomere dysfunction-based models of accelerated aging (Terc^{-/-} and Wm^{-/-} Terc^{-/-} mutants) where osteoporosis is a known phenotype. Significant increases in MAR were accompanied by increases in bone turnover and significant increases in normalized osteoid surface and bone volume (FIG. 8). These findings are consistent with a strong anabolic effect where bone resorption is coupled to enhanced deposition of mineralized matrix.

TABLE 1
Identification of LMMS-responsive
secretory proteins in human MPCs.

| Molecular | | | Highest relative |
|---|---|---|---|
| weight* | | Unique | expression |
| (kDa) | Gene | Peptides | (Fold) |
| 19 | tissue inhibitor of | 4 | 12.5 |
| | metalloproteinases | | |
| 29 | R-Spondin 1 | 4 | 28.0 |
| 36 | unnamed protein product | 4 | 11.6 |
| | gi62822120 | | |
| 42 | unnamed protein product | 10 | 28.2 |
| | gi189053417 | | |
| 45 | unnamed protein product | 9 | 17.9 |
| | gi189055325 | | |
| 45 | plasminogen activator | 14 | 9.9 |
| | inhibitor-1 precursor | | |
| 61 | unnamed protein product | 6 | 7.2 |
| | gi158258302 | | |
| 129 | unnamed protein product | 6 | 6.4 |
| | gi158256710 | | |
| 139 | Collagen alpha 1 chain | 7 | 8.1 |
| | precursor variant | | |
| 312 | fibrillin-1 precursor | 3 | 4.5 |

*Based on Scaffold 3.0 software analysis. Molecular weights are given for the unprocessed proteins. For unnamed protein products, the National Center for Biotechnology Information (NCBI; http://www.ncbi.nlm.nih.gov/) identifier is given.

### Discussion

A screening strategy has been developed to isolate a new class of genes, referred to as vibration-induced bone-enhancing (vibe) genes, whose protein products are secreted and have the capacity to promote bone formation. By virtue of their secretory status, some vibe proteins are candidates for pre-clinical development as anabolic agents for the treatment of osteoporosis. R-Spondin 1 (RSpo1), a Wnt pathway modulator, has been identified as one such vibe gene. Non-limiting examples of other vibe genes are R-Spondin family members having similar structure and activity, e.g., R-Spondin-2 and R-Spondin-4. The instant Example describes is a characterization of the “vibration secretome” induced by LMMS, with important implications for understanding the response of MPCs to mechanical signals which are transduced by molecular pathways that ultimately lead to new bone formation.

Adaptation to mechanical loading at cortical and cancellous sites is well described.²¹ For example, adaptation to daily, cyclic, axial loading of a long bone results in the inhibition of bone loss, elevated bone mineral content, greater effects at cancellous versus cortical sites, and variation depending on the term and level of loading ²². Disuse or paralysis of limbs show extensive loss of trabecular bone.^{23, 24}

Enhanced external load intensity (amplitude and frequency) and local elevations of strain at resorption cavities can induce bone formation.²⁵ However, with aging the skeleton becomes less responsive to loads. Historically, the osteocyte has been considered the bone cell predominately responsible for the transduction of mechanical signals.²⁶ Located within the bone matrix, osteocytes arise from MPCs through osteoblast differentiation. Osteocyte damage or apoptosis in the young skeleton leads to osteoclastic bone resorption followed by formation, but in the aged skeleton can lead to empty lacunae or micropetrosis where the lacuna fills in with mineral.²⁶ Changes in perilacunar mineral density, elastic modulus of the peri-lacunar matrix, and in the size of lacunae and canaliculi affect mechanosensation by the aging osteocyte.²⁶ Even if the osteocyte remained viable for decades, its mechanoresponsiveness would be compromised. However, other mechanosensitive cells that recognize and respond to forces in the skeleton include MPCs, which respond to LMMS by increasing proliferation and potential to differentiate into osteoblasts.⁹ In the case of Rspo1, it is rapidly secreted and can be found in MPC supernatants as well as in circulation by one hour after stimulation by LMMS. Without being bound to a particular theory, regulation of secretion is the most likely explanation for these observations, and is consistent with the differential expression of secreted (but not cellular) Rspo1 between vibration-induced and uninduced conditions in early-passage cells.

Noninvasive delivery of LMMS improves both quantity and quality of trabecular bone, are anabolic to trabecular bone in children, increase bone and muscle mass in the weight-bearing skeleton of young adult females with low BMD, and increase spinal trabecular bone while keeping visceral fat at baseline levels in young women with osteopenia.⁶⁻⁸ However, 202 healthy postmenopausal women with osteopenia who received whole-body vibration therapy for 12 months did not alter BMD or bone structure.²⁷ Delivery of such therapy involves standing on an oscillating platform which produces vertical accelerations that are transmitted from the feet to the weight bearing skeleton. Since transmission of whole-body vibration depends on its intensity, knee-joint angle, distance from the vibratory source, and dampening by soft tissue,^{28, 29} using circulating (systemic) mediators of mechanical signals for bone loss is appealing, and can overcome these limitations.

R-Spondins are secreted Wnt signaling agonists that regulate embryonic patterning and stem cell proliferation in the intestinal crypt and hair follicle. R-Spondins, including RSpo1, can bind to Lgr4, Lgr5 and Lgr6 in the Frizzled/Lrp Wnt receptor complex suggesting that their activity enhances pleiotropic functions in development and stem cell growth.³⁰⁻³³ Little is known about the effects of RSpo1 in the skeleton, but one report suggests that it is protective against inflammatory bone damage in a mouse model of arthritis,¹⁹ which is a distinct indication from osteoporosis. Given the roles of Wnt signaling in bone remodeling,^{34, 35} the application of R-Spondins for regenerative purposes is promising.

This data indicates that additional vibe genes exist and can be identified in a comprehensive proteome-wide approach, and that, at least among the LMMS-induced secreted proteins differentially expressed between early passage and senescent MPCs, as many as 1 in 80 species is a potential vibe protein. It was previously shown that deficiencies in genome maintenance molecules, such as Werner helicase (Wrn) and telomerase (Terc), are related to a low bone mass phenotype due to impairment in osteogenic potential (decreased MPCs) and osteoblast differentiation (decreased expression of osteoblast markers).³⁶ It was also shown that MPCs derived from Wrn^{-/-} Terc^{-/-} double mutants or Terc^{-/-} single mutants have a reduced in vitro lifespan concomitant with impaired osteogenic potential and osteoblast differentiation, but that telomere dysfunction mediates decreased osteogenesis independent of proliferation.³⁷ Both accelerated aging models were employed in the functional characterization of Rspo1 since they recapitulate many aspects of senile bone loss at early ages, with dysfunctional telomeres as the basis for defects in both proliferation and differentiation in MPCs.

Responsiveness to Rspo1, in terms of promoting bone acquisition, in both Terc^{-/-} and Wrn^{-/-} Terc^{-/-} mutants, was observed. Without wishing to be bound by any particular theory, the response suggests that telomere dysfunction, or other stresses (e.g., DNA damage) leading to the same cellular consequences as telomere dysfunction (e.g., cellular senescence), may be operational in normal skeletal aging. Outside of its role in contributing to telomerase activity, mouse Telomerase Reverse Transcriptase (mTert) has been reported to serve other functions (e.g., to physically occupy gene promoters of Wnt-dependent genes) and to thus serve as a transcriptional modulator of the Wnt signaling pathway.⁵ Wnt signaling leads to upregulation of mTert gene via cooperation between β-catenin and Klf4.⁵¹ Without wishing to be bound by any particular theory, the implications of this are that Wnt agonists (such as Rspo1) may promote bone formation in old wild-type mice by promoting the up-regulation of mTert in mTerc-deficient mice, thus contributing to mTert function(s) that do not depend on telomerase activity. It is also possible that Rspo1 is involved in multiple mechanisms that preserve bone structure, including telomerase-independent functions of Tert such as preservation of sternness in mesenchymal precursors, upregulation of growth factor receptor expression and cell proliferation, as well as regulation of Wnt target genes.⁵²⁻⁵⁵

If functional deficits in osteoblasts that occur with aging play a major role in the uncoupling of bone formation and resorption, then recruitment of osteoblast precursors and osteoblast differentiation become critical components in maintaining skeletal homeostasis. Aging effects on human MPCs manifest as declines in measures associated with osteogenic potential, particularly after the age of forty.¹⁰⁻¹⁸ MPCs from aged donors also tend to have decreased proliferative potential.¹⁸ Interestingly, many changes in gene expression that occur with senescence appear to be unrelated to growth arrest, especially in fibroblasts.³⁸ For example, many senescent cells overexpress genes that encode secreted proteins that can alter the tissue microenvironment, including proteins that remodel the extracellular matrix or mediate local inflammation.³⁹⁻⁴³ Interestingly, regulation of secretion may also account for the differential expression of secreted Rspo1 between vibration-induced and uninduced conditions in early-passage cells, as well as between early- and late-passage (senescent) cells after LMMS. Without wishing to be bound by any particular theory, these findings raise the possibility that as senescent cells increase in number with age, they might contribute to age-related decrements in tissue structure and function. This senescence-associated secretory phenotype is also evident in MPCs and this data indicates that it results in the diminished secretion of LMMS-induced proteins, a finding that can be exploited to identify other vibe genes.

### REFERENCES

1. Rubin, J, Rubin, C, and Jacobs, C R (2006). Molecular pathways mediating mechanical signaling in bone. Gene 367: 1-16.
2. Dalsky, G P, Stocke, K S, Ehsani, A A, Slatopolsky, E, Lee, W C, and Birge, S J, Jr. (1988). Weight-bearing exercise training and lumbar bone mineral content in postmenopausal women. Ann Intern Med 108: 824-828.
3. Smith, E L, Gilligan, C, McAdam, M, Ensign, C P, and Smith, P E (1989). Deterring bone loss by exercise intervention in premenopausal and postmenopausal women. Calcif Tissue Int 44: 312-321.
4. Feskanich, D, Willett, W, and Colditz, G (2002). Walking and leisure-time activity and risk of hip fracture in postmenopausal women. JAMA 288: 2300-2306.
5. Wiswell, R A, Hawkins, S A, Dreyer, H C, and Jaque, S V (2002). Maintenance of BMD in older male runners is independent of changes in training volume or VO(2)peak. J Gerontol A Biol Sci Med Sci 57: M203-208.
6. Rubin, C, Turner, A S, Muller, R, Mittra, E, McLeod, K, Lin, W, et al. (2002). Quantity and quality of trabecular bone in the femur are enhanced by a strongly anabolic, noninvasive mechanical intervention. J Bone Miner Res 17: 349-357.
7. Ward, K, Alsop, C, Caulton, J, Rubin, C, Adams, J, and Mughal, Z (2004). Low magnitude mechanical loading is osteogenic in children with disabling conditions. J Bone Miner Res 19: 360-369.
8. Gilsanz, V, Wren, T A, Sanchez, M, Dorey, F, Judex, S, and Rubin, C (2006). Low-level, high-frequency mechanical signals enhance musculoskeletal development of young women with low BMD. J Bone Miner Res 21: 1464-1474.
9. Luu, Y K, Capilla, E, Rosen, C J, Gilsanz, V, Pessin, J E, Judex, S, et al. (2009). Mechanical stimulation of mesenchymal stem cell proliferation and differentiation promotes osteogenesis while preventing dietary-induced obesity. J Bone Miner Res 24: 50-61.
10. D'Ippolito, G, Schiller, P C, Ricordi, C, Roos, B A, and Howard, G A (1999). Age-related osteogenic potential of mesenchymal stromal stem cells from human vertebral bone marrow. J Bone Miner Res 14: 1115-1122.
11. Erdmann, J, Kogler, C, Diel, I, Ziegler, R, and Pfeilschifter, J (1999). Age-associated changes in the stimulatory effect of transforming growth factor beta on human osteogenic colony formation. Mech Ageing Dev 110: 73-85.
12. Long, M W, Ashcraft, E K, Normalle, D, and Mann, K G (1999). Age-related phenotypic alterations in populations of purified human bone precursor cells. J Gerontol A Biol Sci Med Sci 54: B54-62.
13. Mueller, S M, and Glowacki, J (2001). Age-related decline in the osteogenic potential of human bone marrow cells cultured in three-dimensional collagen sponges. J Cell Biochem 82: 583-590.
14. Muschler, G F, Nitto, H, Boehm, C A, and Easley, K A (2001). Age- and gender-related changes in the cellularity of human bone marrow and the prevalence of osteoblastic progenitors. J Orthop Res 19: 117-125.
15. Nishida, S, Endo, N, Yamagiwa, H, Tanizawa, T, and Takahashi, H E (1999). Number of osteoprogenitor cells in human bone marrow markedly decreases after skeletal maturation. J Bone Miner Metab 17: 171-177.
16. Oreffo, R O, Bord, S, and Triffitt, J T (1998). Skeletal progenitor cells and ageing human populations. Clin Sci (Lond) 94: 549-555.
17. Stenderup, K, Justesen, J, Eriksen, E F, Rattan, S I, and Kassem, M (2001). Number and proliferative capacity of osteogenic stem cells are maintained during aging and in patients with osteoporosis. J Bone Miner Res 16: 1120-1129.
18. Zhou, S, Greenberger, J S, Epperly, M W, Goff, J P, Adler, C, Leboff, M S, et al. (2008). Age-related intrinsic changes in human bone-marrow-derived mesenchymal stem cells and their differentiation to osteoblasts. Aging Cell 7: 335-343.
19. Kronke, G, Uderhardt, S, Kim, K A, Stock, M, Scholtysek, C, Zaiss, M M, et al. (2010). R-spondin 1 protects against inflammatory bone damage during murine arthritis by modulating the Wnt pathway. Arthritis Rheum 62: 2303-2312.
20. Hankenson, K D, Sweetwyne, M T, shitaye, H, and Posey, K L (2010). Thrombospondins and novel TSR-containing proteins, R-spondins, regulate bone formation and remodeling. Curr Osteoporos Rep 8: 68-76.
21. Robling, A G, and Turner, C H (2009). Mechanical signaling for bone modeling and remodeling. Crit Rev Eukaryot Gene Expr 19: 319-338.
22. Fritton, J C, Myers, E R, Wright, T M, and van der Meulen, M C (2005). Loading induces site-specific increases in mineral content assessed by microcomputed tomography of the mouse tibia. Bone 36: 1030-1038.
23. Eser, P, Frotzler, A, Zehnder, Y, Wick, L, Knecht, H, Denoth, J, et al. (2004). Relationship between the duration of paralysis and bone structure: a pQCT study of spinal cord injured individuals. Bone 34: 869-880.
24. Shields, R K, Dudley-Javoroski, S, Boaldin, K M, Corey, T A, Fog, D B, and Ruen, J M (2006). Peripheral quantitative computed tomography: measurement sensitivity in persons with and without spinal cord injury. Arch Phys Med Rehabil 87: 1376-1381.
25. Huiskes, R, Ruimerman, R, van Lenthe, G H, and Janssen, J D (2000). Effects of mechanical forces on maintenance and adaptation of form in trabecular bone. Nature 405: 704-706.
26. Bonewald, L F (2011). The amazing osteocyte. J Bone Miner Res 26: 229-238.
27. Slatkovska, L, Alibhai, S M, Beyene, J, Hu, H, Demaras, A, and Cheung, A M (2011). Effect of 12 months of whole-body vibration therapy on bone density and structure in postmenopausal women: a randomized trial. Ann Intern Med 155: 668-679, W205.
28. Kiiski, J, Heinonen, A, Jarvinen, T L, Kannus, P, and Sievanen, H (2008). Transmission of vertical whole body vibration to the human body. J Bone Miner Res 23: 1318-1325.
29. Rubin, C, Pope, M, Fritton, J C, Magnusson, M, Hansson, T, and McLeod, K (2003). Transmissibility of 15-hertz to 35-hertz vibrations to the human hip and lumbar spine: determining the physiologic feasibility of delivering low-level anabolic mechanical stimuli to skeletal regions at greatest risk of fracture because of osteoporosis. Spine (Phila Pa 1976) 28: 2621-2627.
30. Birchmeier, W (2011). Stem cells: Orphan receptors find a home. Nature 476: 287-288.
31. Carmon, K S, Gong, X, Lin, Q, Thomas, A, and Liu, Q (2011). R-spondins function as ligands of the orphan receptors LGR4 and LGR5 to regulate Wnt/beta-catenin signaling. Proc Natl Acad Sci USA 108: 11452-11457.
32. de Lau, W, Barker, N, Low, T Y, Koo, B K, L i, V S, Teunissen, H, et al. (2011). Lgr5 homologues associate with Writ receptors and mediate R-spondin signalling. Nature 476: 293-297.
33. Glinka, A, Dolde, C, Kirsch, N, Huang, Y L, Kazanskaya, O, Ingelfinger, D, et al. (2011). LGR4 and LGR5 are R-spondin receptors mediating Wnt/beta-catenin and Wnt/PCP signalling. EMBO Rep 12: 1055-1061.
34. Krishnan, V, Bryant, H U, and Macdougald, O A (2006). Regulation of bone mass by Wnt signaling. J Clin Invest 116: 1202-1209.
35. Wagner, E R, Zhu, G, Zhang, B Q, Luo, Q, Shi, Q, Huang, E, et al. (2011). The therapeutic potential of the Wnt signaling pathway in bone disorders. Curr Mol Pharmacol 4: 14-25.
36. Pignolo, R J, Suda, R K, McMillan, E A, Shen, J, Lee, S H, Choi, Y, et al. (2008). Defects in telomere maintenance molecules impair osteoblast differentiation and promote osteoporosis. Aging Cell 7: 23-31.
37. Wang, H, Chen, Q, Lee, S H, Choi, Y, Johnson, F B, and Pignolo, RJ (2012). Impairment of Osteoblast Differentiation Due to Proliferation-independent Telomere Dysfunction in Mouse Models of Accelerated Aging. Aging Cell.
38. Campisi, J, and d′Adda di Fagagna, F (2007). Cellular senescence: when bad things happen to good cells. Nat Rev Mol Cell Biol 8: 729-740.
39. Bavik, C, Coleman, I, Dean, J P, Knudsen, 13, Plymate, S, and Nelson, P S (2006). The gene expression program of prostate fibroblast senescence modulates neoplastic epithelial cell proliferation through paracrine mechanisms. Cancer Res 66: 794-802.
40. Coppe, J P, Kauser, K, Campisi, J, and Beausejour, C M (2006). Secretion of vascular endothelial growth factor by primary human fibroblasts at senescence. J Biol Chem 281: 29568-29574,
41. Krtolica, A, Parrinello, S, Lockett, S, Desprez, P Y, and Canapisi, J (2001). Senescent fibroblasts promote epithelial cell growth and tumorigenesis: a link between cancer and aging. Proc Nail Acad Sci USA 98: 12072-12077.
42. Martens, J W, Sieuwerts, A M, Bolt-deVries, J, Bosma, P T, Swiggers, S J, Klijn, J G, et al (2003). Aging of stromal-derived human breast fibroblasts might contribute to breast cancer progression. Thromb Haemost 89: 393-404.
43. Parrinello, S, Coppe, J P, Krtolica, A, and Campisi, J (2005). Stromal-epithelial interactions in aging and cancer: senescent fibroblasts alter epithelial cell differentiation. J Cell Sci 118: 485-496.
44. Du, X, Shen, 3, Kugan, N, Furth, E E, Lombard, D B, Cheung, C, et al. (2004). Telomere shortening exposes functions for the mouse Werner and Bloom syndrome genes. Mol Cell Biol 24: 8437-8446.
45. Fritton, J C, Rubin, C T, Qin, Y X, and McLeod, K J (1997). Whole-body vibration in the skeleton: development of a resonance-based testing device. Ann Biomed Eng 25: 831-839.
46. Wei, Q, Yokota, C, Semenov, M V, Doble, B, Woodgett, J, and He, X (2007). R-spondin 1 is a high affinity ligand for LRP6 and induces LRP6 phosphorylation and beta-catenin signaling. J Biol Chem 282: 15903-15911.
47. Parfitt, A M, Drezner, M K, Glorieux, F H, Kanis, J A, Malluche, H, Meunier, P J, et al. (1987). Bone histomorphometry: standardization of nomenclature, symbols, and units. Report of the ASBMR Histomorphometry Nomenclature Committee. J Bone Miner Res 2: 595-610.
48. Cristofalo V J, Charpentier R (1980) A standard procedure for cultivating human diploid fibroblastlike cells to study cellular aging. Journal of Tissue Culture Methods 6: 117-121.
49. Egan K P, Brennan T A, Pignolo R J (2012) Bone histomorphometry using free and commonly available software. Histopathology 61: 1168-1173.
50. Park J I, Venteicher A S, Hong J Y, Choi J, Jun S, Shkreli M, Chang W, Meng Z, Cheung P, Ji H, McLaughlin M, Veenstra T D, Nusse R, McCrea P D, Artandi S E (2009) Telomerase modulates Wnt signalling by association with target gene chromatin. Nature 460: 66-72.
51. Hoffmeyer K, Raggioli A, Rudloff S, Anton R, Hierholzer A, Del Valle I, Hein K, Vogt R, Kemler R (2012) Wnt/beta-catenin signaling regulates telomerase in stem cells and cancer cells. Science 336: 1549-1554.
52. Barker N, Hurlstone A, Musisi H, Miles A, Bienz M, Clevers H (2001) The chromatin remodelling factor Brg-1 interacts with beta-catenin to promote target gene activation. EMBO J 20: 4935-4943.
53. Beck S, Jin X, Sohn Y W, Kim J K, Kim S H, Yin J, Pian X, Kim S C, Nam D H, Choi Y J, Kim H (2011) Telomerase activity-independent function of TERT allows glioma cells to attain cancer stern cell characteristics by inducing EGFR expression. Mol Cells 31: 9-15.
54. Choi J, Southworth L K, Sarin K Y, Venteicher AS, Ma W, Chang W, Cheung P, Jun S, Artandi M K, Shah N, Kim S K, Artandi S E (2008) TERT promotes epithelial proliferation through transcriptional control of a Myc- and Wnt-related developmental program. PLoS Genet 4: el O.
55. Zhang Y, Toh L, Lau P, Wang X (2012) Human telomerase reverse transcriptase (hTERT) is a novel target of the Wnt/beta-catenin pathway in human cancer. J Biol Chem 287: 32494-32511.

Various publications, patents, and GenBank Accession Numbers are cited herein, the contents of which are hereby incorporated by reference in their entireties.

1. A method of treating or preventing osteoporosis or a bone-related disorder in a subject, comprising administering a therapeutically effective amount of a pharmaceutical composition comprising an RSpo1 agonist to the subject.

2. A method of anabolically increasing bone formation, slowing the decrease of bone mineral density, increasing bone mineral density, or increasing bone mass in a subject, comprising administering a therapeutically effective amount of a pharmaceutical composition comprising an RSpo1 agonist to the subject.

3. The method of claim 1 or 2, wherein the RSpo1 agonist is a recombinant RSpo1 protein or functional fragment thereof.

4. The method of claim 3, wherein the RSpo1 agonist is an RSpo1 protein, or functional fragment thereof, fused to the Fc portion of an antibody, or a portion thereof.

5. The method of claim 1 or 2, wherein the RSpo1 agonist is an RSpo1 peptidomimetic.

6. The method of claim 1 or 2, wherein the RSpo1 agonist is a small molecule.

7. The method of claim 1 or 2, wherein the RSpo1 agonist is a therapeutic vector comprising a nucleic acid molecule encoding RSpo1 protein or a functional fragment thereof.

8. The method of claim 1 or 2, wherein the pharmaceutical composition comprises at least one of a pharmaceutically acceptable diluent, carrier, solubilizer, emulsifier, preservative, and adjuvant.

9. The method of claim 1, wherein the bone-related disorder is osteopenia.

10. A method of diagnosing or aiding in the diagnosis of osteoporosis or a bone-related disorder in a patient, the method comprising comparing: a) the level of RSpo1 in a sample, and b) the normal level of RSpo1 in a control non-osteoporosis or bone-related disorder sample, wherein a level of RSpo1 in the patient sample that is significantly less than the control RSpo1 level is an indication that the patient is afflicted with or predisposed to osteoporosis or a bone-related disorder.

11. The method of claim 10, wherein the samples are obtained from human subjects.

12. The method of claim 10, wherein the presence of RSpo1 is detected using a reagent which specifically binds with an RSpo1 protein or fragment thereof.

13. The method of claim 12, wherein the reagent is selected from the group consisting of an antibody, an antibody derivative, and an antibody fragment.

14. The method of claim 10, further comprising treating said subject for osteoporosis or the bone-related disorder.

15. A method for identifying proteins involved in the formation of bone growth comprising:
(a) mechanically stimulating cells that are sensitive to mechanical stimulation;
(b) isolating proteins secreted from said cells following stimulation;
(c) comparing the levels of proteins expressed from the stimulated cells with those from unstimulated cells,
wherein proteins secreted from the stimulated cells that are expressed at a higher or lower level as compared to proteins expressed from the unstimulated cells are involved in the formation of bone growth.

16. The method of claim 15, further comprising comparing expression of said proteins that are expressed at a higher or lower level in stimulated aged cells versus expression of said proteins in stimulated non-senescent cells.

17. The method of claim 15, wherein proteins that are expressed at a higher level upon stimulation than proteins from the unstimulated cells are capable of increasing bone formation.

18. The method of claim 15, wherein the stimulation is by low magnitude mechanical signals (LMMS).

19. The method of any one of claim 15, 16, or 17, wherein said cells are mesenchymal progenitor cells (MPCs).

20. A pharmaceutical composition comprising an RSpo agonist and a pharmaceutically acceptable carrier for use in the treatment or prevention of osteoporosis or a bone-related disorder in a subject.

21. The pharmaceutical composition of claim 20, wherein the RSpo agonist is a non-naturally-occurring nucleic acid molecule encoding an RSpo protein or functional portion thereof.

22. The pharmaceutical composition of claim 20, wherein the RSpo agonist is an RSpo protein or functional portion thereof.


SEQUENCE LISTING (7 sequences)

SEQ ID NO: 1 - DNA, 2339 bp, Homo sapiens
cgggtcgacgatttcgtcgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacacagacacaagacacacacgctcaaacccctgtccacatatacaaccatacatacttgcacatgtgtgttcatgtacacacgcagacacagacaccacacacacacatacacacacacacacacacgcacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtggcctgggaagaaaccttcttcccatcctgttcagcactcccagctgtgtgactttatcgttggagagtattgttaccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgataatctcaggtctcattccagacctggagagccatgagtctaaattttaagattcctgatgattggcatgttacccaaatttgagaagtgct

SEQ ID NO: 2 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 3 - DNA, 3149 bp, Homo sapiens
agcctagacttagatgccttggaccacagcaccacctacttatagaagcatcccaagcctcagccggtctgcatctccatcggaaagtgcgcttgccacatcccttcggatcacttcgtcctcccgagagcgttctgccttctacagctcggaaagaaagaaatcttagctgtgaagtgaccgtggagaaagcgcaggaagcgacacaattggttagggaggcagagagtgtgagcgggcgcaccccttgcctggggaccgcgctcgcgggcggggacggagcatcccagtggctgcacccgccgctccgcgctcctgcctggcgtcgccaaccccgcggcggccgctggaattccagagctgccaggcgctcccagccggtctcggcaaacttttccccagcccacgtgctaaccaagcggctcgcttcccgagcccgggatggagcaccgcgcctagggaggccgcgccgcccgagacgtgcgcacggttcgtggcggagagatgctgatcgcgctgaactgaccggtgcggcccgggggtgagtggcgagtctccctctgagtcctccccagcagcgcggccggcgccggctctttgggcgaaccctccagttcctagactttgagaggcgtctctcccccgcccgaccgcccagatgcagtttcgccttttctcctttgccctcatcattctgaactgcatggattacagccactgccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacacggcaaattgttaaaaagccagtgaaagacacaatactgtgtccaaccattgctgaatccaggagatgcaagatgacaatgaggcattgtccaggagggaagagaacaccaaaggcgaaggagaagaggaacaagaaaaagaaaaggaagctgatagaaagggcccaggagcaacacagcgtcttcctagctacagacagagctaaccaataaaacaagagatccggtagatttttaggggtttttgtttttgcaaatgtgcacaaagctactctccactcctgcacactggtgtgcagcctttgtgctgctctgcccagtatctgttcccagtaacatggtgaaaggaagcaccaccagcatggcccctgtgttatttatgctttgatttgaatctggagactgtgaaggcaggagtaagtgcacagcccgtgacttggctcagtgtgtgctgagagaatccgtccccggcaccatggacatgctagaggtgtgaggctgcagaacaccgctggaggacggacttgtgcctatttatgtgaaagaagatgcttggcaggcaatgcgctactcactcgtgacctttatttctcacattgtgcattttcaaggatatgtttgtgtggatatctgcttagtgttaccacatggtattctcagcatgttaccttcacactgttgtgcgatgaaactgcttttagctgaggatatgctctggaaattcctgctcagtttcactgcagccctaatatgtacatatactgcaggagctacatataaagctcttatttactgtatatttatgctttcttgtgggtaacaagtcatacctgattaatatgatgccactttgtttctagtggttcctaacccattgtctgataaatgacttttctagtttggggaattgacacttgttttgttgcctcttgaaactttttttttttcccctcattgtgggcttatttctcattgtaagggtaggataaactagtttttgtatatagagtcaaatgaccagtgtcaaagagtttgcatattgggtagaccttctccactccacatgtcccacacatatagataaagcagcaggcggcatctggcaatcagaagcccaaactgcctttgagtctaagatgtgatgactttgatgaaacacaactgaaaacatgagggactatatccagtcacttgtagccagtttcacaggccagctacagaattgtccaaacaaacattatttctgactgcaatttttttcccccaaatttaaagcaatccctggctttaaatgacaaggcacctaccaatgttcttgggtcactgaagaagctactaccatgagcctgggcatagaattttaggagataaaaggatgaatttctgtgactgccagtcagatcttaacaggtttctgttgagccagaatctgtttcagatccaagatggagaggaacactatggaaacttcccaggtgactttcagagcagttgtttcaaacacatcattgtccttttaggggaaccagtttttagaaggttgtgaattggctttttcacaaagcatgattatcttcctggctgatccaggagaaaattagaacagaaaaataatggttgtggattttgaaacaaagcaaggtaaagccttttttttttcaccttgcattggcaaaactacctcttcagtgtttttaacttttgattcaaaagcatcttaccaataaggataaatatcatatacatcgttatgaaaatattgctatgagataataagccacatatgaatgttgtatacaactttagggtttacatttaatcctgaagtgttacctcctttcatgtctatttacactattttcccatttactaagtggggagggggtctccttatatagtgcttcatcgttaataagtcaatacctgttgttcctgggatgttcttttttgtgcattaaaaacttcaaaatta

SEQ ID NO: 4 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 5 - DNA, 2725 bp, Homo sapiens
gcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaaaaaaaaaaaaaaaaa

SEQ ID NO: 6 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK